(19) United States
(12) Patent Application Publication
(10) Pub. No.: US 2026/0253681 A1
(43) Pub. Date: Aug. 27, 2026

(54) MOLECULAR GENERATION

(71) Applicant: Tencent Technology (Shenzhen) Company Limited, Shenzhen (CN)

(72) Inventors: Tingyang XU, Shenzhen (CN); Tian BIAN, Shenzhen (CN)

(73) Assignee: Tencent Technology (Shenzhen) Company Limited, Shenzhen (CN)

(21) Appl. No.: 19/653,091

(22) Filed: Apr. 20, 2026

Related U.S. Application Data
(63) Continuation of application No. PCT/CN2025/078686, filed on Feb. 24, 2025.

(30) Foreign Application Priority Data
Feb. 26, 2024 (CN) 202410210835.3

(51) Int. Cl. G16C 20/50; G06F 30/27; G16C 20/70

(52) U.S. Cl. CPC G16C 20/50; G16C 20/70; G06F 30/27

(57) ABSTRACT

Initial hierarchical embeddings that include an initial molecular graph embedding, an initial subgraph embedding, and an initial atomic embedding, are generated. An initial molecular graph includes initial nodes and initial edges, the initial nodes represent initial atoms, the initial edges represent chemical bonds connecting the initial atoms, and each initial subgraph represents a regional molecular structure of the initial molecular graph. Noises are removed from the initial hierarchical embeddings to obtain generated hierarchical embeddings, the noises are predicted based on a machine learning model that includes learned relationships among molecular graphs, subgraphs, and atoms. The generated hierarchical embeddings are decoded into molecular structure information of a generated molecule, and the molecular structure information includes atom types of used atoms in the generated molecule and chemical bond types of inter-atomic chemical bonds connecting the used atoms in the generated molecule. Apparatus and non-transitory computer-readable storage medium counterpart embodiments are also contemplated.

### RELATED APPLICATIONS

The present application is a continuation of International Application No. PCT/CN2025/078686, filed on Feb. 24, 2025, which claims priority to Chinese Patent Application No. 202410210835.3, filed on Feb. 26, 2024. The entire disclosures of the prior applications are hereby incorporated by reference.

### FIELD OF THE TECHNOLOGY

Embodiments of this disclosure relate to the field of artificial intelligence technologies, including a molecular generation method and apparatus, a device, and a storage medium.

### BACKGROUND OF THE DISCLOSURE

A molecular generation task is to generate a molecule having desired properties by designing a synthetic route according to a given molecular property or structure. With the development of chemical synthesis and materials science, the molecular generation task becomes increasingly important. In the related technology, a heuristic search algorithm or a rule-based method is used to generate a new molecule based on a known molecule, a search strategy is used to modify the known molecule based on a molecular transformation rule, thus generating a new molecule. However, the solutions provided in the related technology are usually limited by the size of a search space and the calculation complexity. As a result, the quantity of molecules that can be generated is limited.

### SUMMARY

Embodiments of this disclosure provide a molecular generation method and apparatus, a device, and a storage medium.

Some aspects of the disclosure provide a method of molecular generation. For example, initial hierarchical embeddings are generated, the initial hierarchical embeddings include an initial molecular graph embedding of an initial molecular graph, an initial subgraph embedding of one or more initial subgraphs of the initial molecular graph, and an initial atomic embedding of initial atoms. The initial molecular graph includes initial nodes and initial edges, the initial nodes represent the initial atoms, the initial edges represent chemical bonds connecting the initial atoms, and each of the one or more initial subgraphs represents a regional molecular structure of the initial molecular graph. Noises are removed from the initial hierarchical embeddings to obtain generated hierarchical embeddings, the noises are predicted based on a machine learning model that includes learned relationships among molecular graphs, subgraphs, and atoms. The generated hierarchical embeddings include a generated molecular graph embedding, a generated subgraph embedding, and a generated atomic embedding of generated hierarchical embeddings. The generated hierarchical embeddings are decoded into molecular structure information of a generated molecule, and the molecular structure information includes atom types of used atoms in the generated molecule and chemical bond types of inter-atomic chemical bonds connecting the used atoms in the generated molecule.

Some aspects of the disclosure provide an apparatus of molecular generation. The apparatus includes processing circuitry configured to generate initial hierarchical embeddings, the initial hierarchical embeddings include an initial molecular graph embedding of an initial molecular graph, an initial subgraph embedding of one or more initial subgraphs of the initial molecular graph, and an initial atomic embedding of initial atoms. The initial molecular graph includes initial nodes and initial edges, the initial nodes represent the initial atoms, the initial edges represent chemical bonds connecting the initial atoms, and each of the one or more initial subgraphs represents a regional molecular structure of the initial molecular graph. Noises are removed from the initial hierarchical embeddings to obtain generated hierarchical embeddings, the noises are predicted based on a machine learning model that includes learned relationships among molecular graphs, subgraphs, and atoms, the generated hierarchical embeddings include a generated molecular graph embedding, a generated subgraph embedding, and a generated atomic embedding of generated hierarchical embeddings. The generated hierarchical embeddings are decoded into molecular structure information of a generated molecule, the molecular structure information includes atom types of used atoms in the generated molecule and chemical bond types of inter-atomic chemical bonds connecting the used atoms in the generated molecule.

Some aspects of the disclosure provide a non-transitory computer-readable storage medium storing instructions which when executed by at least one processor cause the at least one processor to perform methods of molecular generation in the present disclosure.

The molecular generation method in this embodiment of this disclosure may include: generating random initial hierarchical embeddings, the initial hierarchical embeddings including an initial graph embedding (also referred to as initial molecular graph embedding in some examples) representing a molecular graph, an initial subgraph embedding representing a subgraph, and an initial atomic embedding representing atoms, the molecular graph being composed of nodes and edges, the nodes representing the atoms, the edges representing chemical bonds connecting the atoms, and the subgraph representing a regional molecular structure of the molecular graph; predicting and removing noises from the initial molecular graph embedding, the initial subgraph embedding, and the initial atomic embedding by using a trained machine learning model and a learned relationship among the molecular graph, the subgraph, and the atoms, to obtain a generated molecular graph embedding, a generated subgraph embedding, and a generated atomic embedding of generated hierarchical embeddings; and decoding the generated hierarchical embeddings into molecular structure information of a generated molecule, the molecular structure information including atom types of atoms in the generated molecule and chemical bond types of inter-atomic chemical bonds.

A molecular generation apparatus of this implementation of this disclosure may include: an initial sampling module configured to generate random initial hierarchical embeddings, the initial hierarchical embeddings including an initial molecular graph embedding representing a molecular graph, an initial subgraph embedding representing a subgraph, and an initial atomic embedding representing atoms, the molecular graph being composed of nodes and edges, the nodes representing the atoms, the edges representing chemical bonds connecting the atoms, and the subgraph representing a regional molecular structure of the molecular graph; a denoising module configured to predict and remove noises from the initial molecular graph embedding, the initial subgraph embedding, and the initial atomic embedding by using a trained machine learning model and a learned relationship among the molecular graph, the subgraph, and the atoms, to obtain a generated molecular graph embedding, a generated subgraph embedding, and a generated atomic embedding of generated hierarchical embeddings; and a decoding module configured to decode the generated hierarchical embeddings into molecular structure information of a generated molecule, the molecular structure information including atom types of atoms in the generated molecule and chemical bond types of inter-atomic chemical bonds.

A computer device in this embodiment of this disclosure includes a processor (an example of processing circuitry) and a memory. The memory has at least one program stored therein, and the at least one program is loaded and executed by the processor to implement the molecular generation method of the embodiments.

The computer-readable storage medium (e.g., non-transitory computer-readable storage medium) in this embodiment of this disclosure has at least one program stored therein, and the at least one program may be loaded and executed by a processor to implement the molecular generation method of the embodiments.

A computer program product or a computer program in this embodiment of this disclosure is provided. The computer program product or computer program includes at least one program. The at least one program is stored in a computer-readable storage medium. A processor of a computer device reads the at least one program from the computer-readable storage medium, and the processor executes the at least one program to cause the computer device to perform the molecular generation method of the embodiments.

In the embodiments of this disclosure, denoising is performed on different hierarchies of a molecular structure by using the machine learning model based on the learned relationship among the molecular graph, the subgraph, and the atoms, thereby more fully using a mutual constraint relationship between hierarchical structures inside the molecule. This approach is conducive to increasing the quantity of potential generable molecules and enhancing diversity of generable molecules. In addition, by using the subgraph embedding, the machine learning model can determine regional structure information of the molecule based on the subgraph. Compared with using structure information of the entire molecular graph to perform various calculations, using the regional structure information is conducive to lowering a storage requirement in a processing process of the machine learning model and reducing time consumption, thereby facilitating large-scale molecular generation.

### BRIEF DESCRIPTION OF THE DRAWINGS

FIG. 1A shows a schematic diagram of an implementation environment according to an example embodiment of this disclosure.

FIG. 1B shows a flowchart of a molecular generation method according to an embodiment of this disclosure.

FIG. 1C shows a schematic diagram of a structure of a hierarchical denoising network according to an example embodiment of this disclosure.

FIG. 2 shows a flowchart of a molecular generation method according to an example embodiment of this disclosure.

FIG. 3 shows a schematic diagram of hierarchies of a molecular structure according to an example embodiment of this disclosure.

FIG. 4 shows a schematic diagram of N denoising operations according to an example embodiment of this disclosure.

FIG. 5 shows a flowchart of a denoising process according to an example embodiment of this disclosure.

FIG. 6 shows a flowchart of performing a denoising process by a hierarchical denoising network according to an example embodiment of this disclosure.

FIG. 7 shows a schematic diagram of a structure of a hierarchical block according to an example embodiment of this disclosure.

FIG. 8 shows a schematic diagram of a structure of a hierarchical denoising network according to an example embodiment of this disclosure.

FIG. 9 shows a flowchart of a process of generating molecular structure information according to an example embodiment of this disclosure.

FIG. 10 shows a schematic diagram of a structure of a hierarchical denoising network according to another example embodiment of this disclosure.

FIG. 11 shows a schematic diagram of a structure of a molecular generation model according to an example embodiment of this disclosure.

FIG. 12 shows a schematic diagram of a structure of a molecular generation model in a training process according to an example embodiment of this disclosure.

FIG. 13 shows a block diagram of a structure of a molecular generation apparatus according to an example embodiment of this disclosure.

FIG. 14 is a schematic diagram of a structure of a computer device provided according to an example embodiment of this disclosure.

### DETAILED DESCRIPTION

The following describes technical solutions in embodiments of this disclosure with reference to the accompanying drawings. The described embodiments are some of the embodiments of this disclosure rather than all of the embodiments. Other embodiments are within the scope of this disclosure.

Descriptions of terms in this disclosure are provided as examples only and are not intended to limit the scope of the disclosure.

Molecular generation refers to generation of a generated molecule having desired properties or a desired structure by designing a synthetic route, and has wide application in fields such as drug discovery, material design, and catalyst development.

A molecular generation solution provided in the embodiments of this disclosure involves a machine learning technology of artificial intelligence. The molecular generation solution is described through the following embodiments.

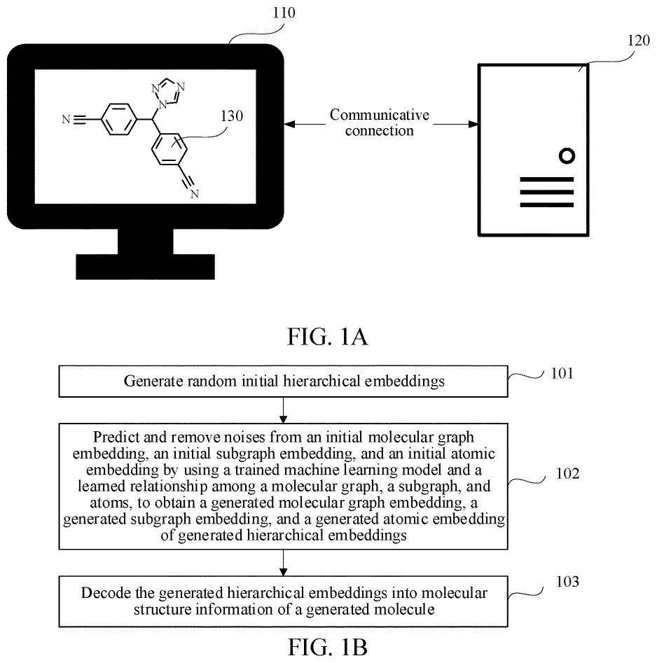

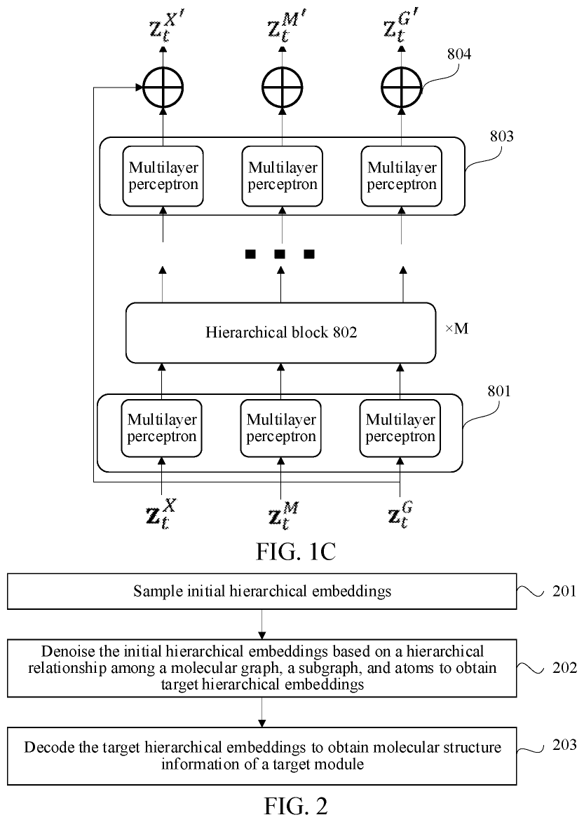

FIG. 1A shows a schematic diagram of an implementation environment according to an example embodiment of this disclosure. The implementation environment may include a terminal 110 and a server 120. The terminal 110 performs data communication with the server 120 through a communication network. In some embodiments, the communication network may be a wired network or a wireless network, and the communication network may be at least one of a local area network, a metropolitan area network, or a wide area network.

The terminal 110 may be an electronic device, for example, a mobile terminal such as a smartphone, a tablet computer, or a laptop portable notebook computer, or a desktop computer. There may be one or more terminals. This is not limited in this embodiment of this disclosure. The server 120 is at least one electronic device, for example, may be an independent physical server, or may be a server cluster or a distributed system formed by a plurality of physical servers, or may be a cloud server that provides basic cloud computing services such as a cloud service, a cloud database, cloud computing, a cloud function, cloud storage, a network service, cloud communication, a middleware service, a domain name service, a security service, a content delivery network (CDN), big data, and an artificial intelligence platform.

The solutions provided in this disclosure may be independently completed by the terminal 110 or the server 120, or may be cooperatively completed by the terminal 110 and the server 120.

In an implementation example, the solutions provided in the embodiments of this disclosure are performed by a molecular generation model. The molecular generation model is a machine learning model that is trained to perform a molecular generation task. In some embodiments, the molecular generation model is deployed in the terminal 110. In a case that a molecular generation instruction is received, the terminal 110 generates initial hierarchical embeddings, uses the molecular generation model to denoise the initial hierarchical embeddings, to obtain generated hierarchical embeddings (also referred to as target hierarchical embeddings), and decodes the generated hierarchical embeddings, to obtain molecular structure information of a generated molecule (also referred to as a target molecule). The terminal 110 may present, to a user through a display screen component, a generated molecule 130 corresponding to the molecular structure information.

In some embodiments, the molecular generation model is deployed in the server 120. In a case that a molecular generation instruction is received, the terminal 110 transmits the molecular generation instruction to the server 120. The server generates initial hierarchical embeddings in a case of receiving the molecular generation instruction, uses the molecular generation model to denoise the initial hierarchical embeddings based on a hierarchical relationship among a molecular graph, a subgraph, and atoms, to obtain generated hierarchical embeddings, and then obtains molecular structure information of a generated molecule based on the generated hierarchical embeddings. The server 120 returns the molecular structure information of the generated molecule to the terminal 110 after obtaining the molecular structure information of the generated molecule.

In some embodiments, in a case of receiving a molecular generation instruction, the terminal 110 generates initial hierarchical embeddings, and transmits the initial hierarchical embeddings to the server 120. The server 120 denoises the initial hierarchical embeddings and decodes obtained generated hierarchical embeddings, to obtain molecular structure information which is returned to the terminal 110.

For ease of description, the following embodiments use an example in which a molecular generation method is performed by a computer device for description. The computer device in the embodiments may be the terminal 110, or the server 120, or both the terminal 110 and the server 120.

FIG. 1B shows a flowchart of a molecular generation method according to an embodiment of this disclosure. As shown in FIG. 1B, the exemplary method may include the following operations:

Operation 101: Generate random initial hierarchical embeddings.

The initial hierarchical embeddings are initial data for a molecular generation task. The machine learning model performs a series of modifications on inputted initial hierarchical embeddings to finally obtain hierarchical embeddings (also referred to as generated hierarchical embeddings or target hierarchical embeddings) of a generated new molecule (hereinafter referred to as a generated molecule or a target molecule). The generated hierarchical embeddings may be decoded into structure information of the generated molecule. The initial hierarchical embeddings in the embodiments include an initial molecular graph embedding representing a molecular graph, an initial subgraph embedding representing a subgraph, and an initial atomic embedding representing atoms. The molecular graph (also referred to as a graph) represents a complete structure of a molecule, and the subgraph represents a regional molecular structure of the molecular graph. Both the molecular graph and the subgraph may be composed by nodes and edges. The nodes represent the atoms, and the edges represent chemical bonds connecting the atoms.

The initial hierarchical embeddings may be generated in any manner, and the generated initial hierarchical embeddings comply with a preset distribution manner, for example, a Gaussian distribution. In some embodiments, the initial graph embedding, the initial subgraph embedding, and the initial atomic embedding may be respectively sampled from a preset candidate molecular graph embedding set, a candidate subgraph embedding set, and a candidate atomic embedding set, the initial graph embedding, the initial subgraph embedding, and the initial atomic embedding complying with a Gaussian distribution. In some embodiments, sampled values of at least two parameters that are preset may be sampled from preset parameter value sets respectively corresponding to the at least two parameters that are preset. Initial molecular structure information is generated based on the sampled values of the at least two parameters, and the initial molecular structure information is transformed into the initial graph embedding, the initial subgraph embedding, and the initial atomic embedding, the at least two parameters complying with the Gaussian distribution.

Operation 102: Predict and remove noises from the initial molecular graph embedding, the initial subgraph embedding, and the initial atomic embedding by using a trained machine learning model and a learned relationship among the molecular graph, the subgraph, and the atoms, to obtain a generated molecular graph embedding, a generated subgraph embedding, and a generated atomic embedding of generated hierarchical embeddings (also referred to as target hierarchical embeddings).

Herein, “noise” refers to redundant information in the initial hierarchical embeddings compared with the generated hierarchical embeddings outputted by the machine learning model. The redundant information is not pre-determined and added into the initial hierarchical embeddings, but is determined by the machine learning model by using a built-in algorithm based on the learned relationship among the molecular graph, the subgraph, and the atoms. For example, the noises in the initial molecular graph embedding, the initial subgraph embedding, or the initial atomic embedding may be predicted by using at least two of the initial molecular graph embedding, the initial subgraph embedding, and the initial atomic embedding. That is, the noise in each of the initial molecular graph embedding, the initial subgraph embedding, or the initial atomic embedding is determined by using at least two of the initial molecular graph embedding, the initial subgraph embedding, and the initial atomic embedding. In the embodiments, noises in different types of embeddings may be determined by different combinations of at least two types of embeddings. For example, the noise in the initial atomic embedding may be predicted by using the initial molecular graph embedding and the initial atomic embedding. The noise in the initial subgraph embedding may be predicted by using the initial subgraph embedding and the initial atomic embedding. The noise in the initial molecular graph embedding may be predicted by using the initial molecular graph embedding, the initial subgraph embedding, and the initial atomic embedding.

Operation 103: Decode the generated hierarchical embeddings into molecular structure information of a generated molecule.

The molecular structure information includes atom types of atoms in the generated molecule and chemical bond types of inter-atomic chemical bonds.

As can be seen, denoising is performed on different hierarchies of a molecular structure, thereby more fully using a mutual constraint relationship between hierarchical structures inside the molecule. This approach is conducive to increasing the quantity of potential generable molecules and enhancing diversity of generable molecules. In addition, by using the subgraph embedding, the machine learning model can determine regional structure information of the molecule based on the subgraph. Compared with using structure information of the entire molecular graph to perform various calculations, using the regional structure information is conducive to lowering a storage requirement in a processing process of the machine learning model and reducing time consumption, thereby facilitating large-scale molecular generation.

In the embodiments, to enhance quality of the generated molecule, the generated hierarchical embeddings may be obtained by performing multiple instances of iterative denoising. For example, for the initial hierarchical embeddings, first intermediate hierarchical embeddings may be obtained by predicting and removing the noises from the initial molecular graph embedding, the initial subgraph embedding, and the initial atomic embedding. In a subsequent (n+1)^{th} instance of denoising, (n+1)^{th} intermediate hierarchical embeddings may be obtained by predicting and removing (n+1)^{th} noises from n^{th} intermediate hierarchical embeddings. The n^{th} intermediate hierarchical embeddings include an n^{th} intermediate molecular graph embedding, an n^{th} intermediate subgraph embedding, and an n^{th} intermediate atomic embedding. When a total of N instances of denoising are preset to be performed, in an n^{th} instance of denoising, the generated hierarchical embeddings may be obtained by predicting and removing n^{th} noises from (N−1)^{th} intermediate hierarchical embeddings. N is a preset total number of instances of denoising, and n is a positive integer greater than 0 and less than N.

For example, the foregoing multiple instances of iterative denoising may be implemented in a machine learning model with a structure similar to that shown in FIG. 11. FIG. 11 shows a schematic diagram of a structure of a molecular generation model according to one example embodiment of this disclosure. The molecular generation model may include at least two hierarchical denoising networks 1101. As shown in FIG. 11, a quantity of hierarchical denoising networks 1101 is represented as T. A 1^{st} hierarchical denoising network 1101 obtains an initial atomic embedding

$${z}_{T}^{X} ,$$

an initial subgraph embedding

$${z}_{T}^{M} ,$$

and an initial graph embedding

$${z}_{T}^{G} ,$$

predicts and removes noises from the initial atomic embedding

$${z}_{T}^{X} ,$$

the initial subgraph embedding

$${z}_{T}^{M} ,$$

and the initial graph embedding

$${z}_{T}^{G}$$

to obtain a first intermediate atomic embedding

$${z}_{T - 1}^{X} ,$$

a first intermediate subgraph embedding

$${z}_{T - 1}^{M} ,$$

and a first intermediate graph embedding

$${z}_{T - 1}^{G} ,$$

and inputs them to a 2^{nd} hierarchical denoising network 1101. In this way, the denoising is performed in sequence through T hierarchical denoising networks 1101. A final generated atomic embedding

$${z}_{0}^{X} ,$$

generated subgraph embedding

$${z}_{0}^{M} ,$$

and generated graph embedding

$${z}_{0}^{G}$$

that are generated by a last (i.e. Tth) hierarchical denoising network 1101 are inputted to a decoding network 1102 and are transformed into molecular structure information of a generated molecule.

In this way, by the multiple instances of iterative denoising, the noises in the initial hierarchical embeddings may be gradually removed, thereby lowering a requirement on algorithm accuracy, reducing algorithm complexity, further enhancing a denoising effect, and enhancing quality of the generated molecule.

In the embodiments, the hierarchical denoising network may include a first multilayer perceptron network, a second multilayer perceptron network, and a hierarchical block located between the first multilayer perceptron network and the second multilayer perceptron network. An (n+1)^{th} hierarchical denoising network is used as an example. The first multilayer perceptron network may transform the n^{th} intermediate hierarchical embeddings into n^{th} intermediate hierarchical hidden variables. The n^{th} intermediate hierarchical hidden variables include an n^{th} intermediate molecular graph hidden variable, an n^{th} intermediate subgraph hidden variable, and an n^{th} intermediate atomic hidden variable. The hierarchical block updates the n^{th} intermediate hierarchical hidden variables based on a relationship among the n^{th} intermediate molecular graph hidden variable, the n^{th} intermediate subgraph hidden variable, and the n^{th} intermediate atomic hidden variable, to obtain updated n^{th} intermediate hierarchical hidden variables. The second multilayer perceptron network predicts (n+1)^{th} noises based on the updated n^{th} intermediate hierarchical hidden variables.

In each iterative denoising process, the hierarchical embeddings are transformed into the hidden variables at hierarchies by using the multilayer perceptron network. The hidden variables are updated based on the relationship between the hidden variables. Then, the updated hidden variables are transformed into updated hierarchical embeddings by using the multilayer perceptron network. Thus, the learned relationship between the hierarchies can be used to better understand the hierarchical embeddings, extract and recombine features, enhance a denoising effect, and finally generate a molecule with higher quality.

To enhance noise prediction accuracy, the embodiments provide a structure of a multi-updated hierarchical denoising network. FIG. 1C shows a schematic diagram of a structure of a hierarchical denoising network according to one example embodiment of this disclosure. As shown in FIG. 1C, the hierarchical denoising network includes a first multilayer perceptron network 801, at least two hierarchical blocks 802 connected in series, a second multilayer perceptron network 803, and a denoising module 804.

An (n+1)^{th} hierarchical denoising network is used as an example. The first multilayer perceptron network 801 may transform n^{th} intermediate hierarchical embeddings, including an n^{th} intermediate atomic embedding

$${z}_{t}^{X} ,$$

an n^{th} intermediate subgraph embedding

$${z}_{t}^{M} ,$$

and an n^{th} intermediate molecular graph embedding

$${z}_{t}^{G} ,$$

into n^{th} intermediate hierarchical hidden variables.

A 1^{st} hierarchical block 802 updates the n^{th} intermediate atomic hidden variable based on the n^{th} intermediate atomic hidden variable and the n^{th} intermediate subgraph hidden variable that are outputted by the first multilayer perceptron network, updates the n^{th} intermediate subgraph hidden variable based on the n^{th} intermediate subgraph hidden variable and the n^{th} intermediate atomic hidden variable, and updates the n^{th} intermediate molecular graph hidden variable based on the n^{th} intermediate subgraph hidden variable, the n^{th} intermediate atomic hidden variable, and the n^{th} intermediate molecular graph hidden variable.

An (m+1)^{th} hierarchical block 802 updates, based on an n^{th} intermediate atomic hidden variable and an n^{th} intermediate subgraph hidden variable that are updated by an m^{th} hierarchical block, an n^{th} intermediate atomic hidden variable updated by the m^{th} hierarchical block, updates, based on the n^{th} intermediate subgraph hidden variable and the n^{th} intermediate atomic hidden variable that are updated by the m^{th} hierarchical block, the n^{th} intermediate subgraph hidden variable updated by the m^{th} hierarchical block, and updates, based on the n^{th} intermediate subgraph hidden variable, the n^{th} intermediate atomic hidden variable, and an n^{th} intermediate molecular graph hidden variable that are updated by the m^{th} hierarchical block, the n^{th} intermediate molecular graph hidden variable updated by the m^{th} hierarchical block.

After updating the n^{th} intermediate atomic hidden variable, the n^{th} intermediate subgraph hidden variable, and the n^{th} intermediate molecular graph hidden variable, an m^{th} hierarchical block 802 outputs, to the second multilayer perceptron network, an updated n^{th} intermediate atomic hidden variable, n^{th} intermediate subgraph hidden variable, and n^{th} intermediate molecular graph hidden variable. M is a total number of hierarchical blocks 802, and m is an integer greater than 0 and less than M.

The second multilayer perceptron network 803 predicts (n+1)^{th} noises based on the updated n^{th} intermediate atomic hidden variable, n^{th} intermediate subgraph hidden variable, and n^{th} intermediate molecular graph hidden variable.

The denoising module 804 removes the (n+1)^{th} noises from the n^{th} intermediate atomic embedding

$${z}_{t}^{X} ,$$

the n^{th} intermediate subgraph embedding

$${z}_{t}^{M} ,$$

and the n^{th} intermediate molecular graph embedding

$${z}_{t}^{G}$$

to obtain an updated n^{th} intermediate atomic embedding

$${z}_{t}^{{X}^{′}} ,$$

n^{th} intermediate subgraph embedding

$${z}_{t}^{{M}^{′}} ,$$

and n^{th} intermediate molecular graph embedding

$${z}_{t}^{{G}^{′}} .$$

In this way, in each instance of iterative denoising, the at least two hierarchical blocks are configured to update the intermediate hierarchical hidden variables for multiple times. In each update, mutual constraint relationships between hierarchies of a molecular structure are considered. Such finer multi-hierarchy update processing is conductive to more accurately determining noise, thereby enhancing the denoising effect.

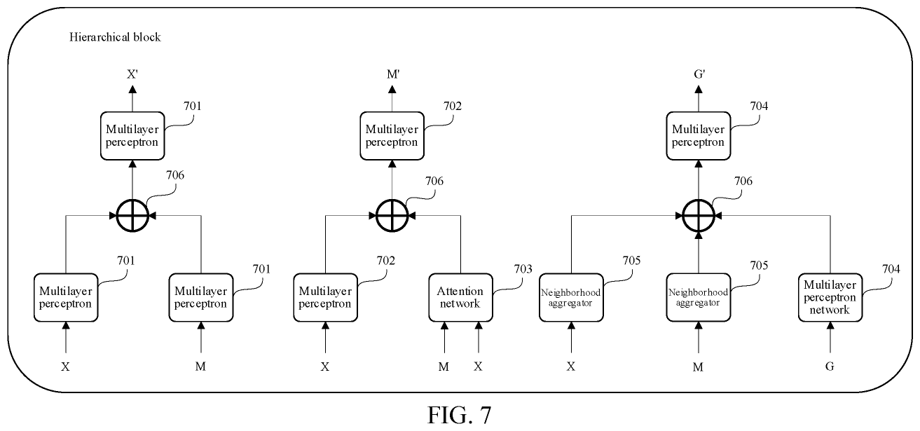

In the embodiments, the hierarchical blocks in the hierarchical denoising network may design processing logics for the hierarchical embeddings based on the relationships between the hierarchies of the molecular structure. FIG. 7 shows a schematic diagram of a structure of a hierarchical block according to one example embodiment of this disclosure. An (m+1)^{th} hierarchical block in an n^{th} hierarchical denoising network is used as an example for description.

The hierarchical block may include at least two third multilayer perceptrons 701 and a fusion module 706. The n^{th} intermediate atomic hidden variable updated by the m^{th} hierarchical block is transformed through a first perceptron among the at least two third multilayer perceptrons 701 to obtain a first transformed hidden variable, and the n^{th} intermediate subgraph hidden variable updated by the m^{th} hierarchical block is transformed through a second perceptron among the at least two third multilayer perceptrons 701 to obtain a second transformed hidden variable. The fusion module 706 fuses the first transformed hidden variable with the second transformed hidden variable to obtain a first fused variable. The first fused variable is transformed through a third perceptron among the at least two third multilayer perceptrons 701 to obtain an n^{th} intermediate atomic hidden variable updated by the (m+1)^{th} hierarchical block.

The hierarchical block may further include at least two fourth multilayer perceptrons 702, an attention network 703, and a fusion module 706. A first attention score is determined through the attention network 703 based on the n^{th} intermediate atomic hidden variable and the n^{th} intermediate subgraph hidden variable that are updated by the m^{th} hierarchical block. The n^{th} intermediate atomic hidden variable updated by the m^{th} hierarchical block is weighted based on the first attention score to obtain a weighted n^{th} intermediate atomic hidden variable. The n^{th} intermediate subgraph hidden variable updated by the m^{th} hierarchical block is transformed through a first perceptron among the at least two fourth multilayer perceptrons 702 to obtain a third transformed hidden variable. The fusion module 706 fuses the third transformed hidden variable with the weighted n^{th} intermediate atomic hidden variable to obtain a second fused variable. The second fused variable is transformed through a second perceptron among the at least two fourth multilayer perceptrons 702 to obtain an n^{th} intermediate subgraph hidden variable updated by the (m+1)^{th} hierarchical block.

The hierarchical block may further include at least two fifth multilayer perceptrons 704, a neighborhood aggregator 705, and a fusion module 706. The n^{th} intermediate molecular graph hidden variable updated by the m^{th} hierarchical block is transformed through a first perceptron among the at least two fifth multilayer perceptrons 704 to obtain a fourth transformed variable. Neighborhood aggregation is performed, through the neighborhood aggregator 705, on the n^{th} intermediate atomic hidden variable updated by the m^{th} hierarchical block to obtain a first aggregated variable. Neighborhood aggregation is performed, through the neighborhood aggregator 705, on the n^{th} intermediate subgraph hidden variable updated by the m^{th} hierarchical block to obtain a second aggregated variable. The fusion module 706 fuses the fourth transformed variable, the first aggregated variable, and the second aggregated variable to obtain a third fused variable. The third fused variable is transformed through a second perceptron among the at least two fifth multilayer perceptrons 704 to obtain an n^{th} intermediate molecular graph hidden variable updated by the (m+1)^{th} hierarchical block.

Through the processing logics of the foregoing hierarchical blocks, the relationships between the hierarchies (the atoms, the subgraph, and the molecular graph) in the molecular structure can be analyzed and understood in a more detailed manner. For example, features in the atomic hidden variables and the subgraph hidden variables are further mined through the multilayer perceptrons, to enhance quality of updated atomic hidden variables. Key associations between the atoms and the molecular structure is caught through the attention network, to enhance quality of the updated hidden variables of the subgraph. Information related to the molecular structure is extracted through the neighborhood aggregator from the atomic hidden variables and the subgraph hidden variables, to update the molecular graph hidden variables, thereby enhancing quality of the updated molecular graph hidden variables.

When there is a preset requirement (which is also referred to as a target molecular property) for molecular properties of the generated molecule, a target molecular property embedding may be inputted to the machine learning model, so as to generate a generated molecule having the target molecular property. The target molecular property may include, for example, at least one of a molecular water solubility and a high syntheticity. The target molecular property may be obtained from another device, for example, an input device associated with the electronic device, and a storage device that can be read by the electronic device. In some embodiments, the target molecular property embedding may be expressed by using a vector s={s₁, s₂, . . . }, where each dimension represents a particular molecular property.

In an embodiment, a sixth multilayer perceptron and a property attention network may be added into the hierarchical denoising network. An n^{th} hierarchical denoising network is used as an example. In the process of obtaining the (n+1)^{th} intermediate hierarchical embeddings by predicting and removing the (n+1)^{th} noises from the n^{th} intermediate hierarchical embeddings, the (n+1)^{th} noises may be removed from the n^{th} intermediate hierarchical embeddings to obtain denoised n^{th} intermediate hierarchical embeddings. A set target molecular property embedding is transformed into a target property hidden space variable through the sixth multilayer perceptron. The (n+1)^{th} intermediate hierarchical embeddings are obtained by updating the denoised n^{th} intermediate hierarchical embeddings through the property attention network based on the target property hidden space variable.

For example, second attention scores of the target property hidden space variable and elements in the denoised n^{th} intermediate hierarchical embeddings may be determined through the property attention network. The second attention scores represent associations between the elements and the target property hidden space variable. Each element includes at least one of the following: an atomic embedding in the n^{th} intermediate atomic embedding; a subgraph embedding element in the n^{th} intermediate subgraph embedding (for example, an element in a dimension in a subgraph embedding vector, which represents one of at least two subgraphs or one element in one subgraph), a node or edge embedding in the n^{th} intermediate molecular graph embedding, and the like. The (n+1)^{th} intermediate hierarchical embeddings are generated by weighting at least one element in the denoised n^{th} intermediate hierarchical embeddings based on the second attention scores.

In this way, in the process of each instance of iterative denoising, the hierarchical embeddings (including the atomic embedding, the subgraph embedding, and the molecular graph embedding) of the hierarchies are updated based on the set target property and the learned relationship between the molecular property and the hierarchies of the molecular structure. For example, weighting performed through the attention network strengthens at least one element in at least one hierarchical embedding. In this way, in the process of the multiple instances of iterative denoising, the molecular structure gradually evolves to a structure with the target molecular property, so that a finally generated molecule has the target molecular property.

To help understand the technical solutions of this disclosure, some example embodiments of various aspects of this disclosure are listed below. Details are merely examples, and the technical solutions of this disclosure are not limited to these details.

FIG. 2 shows a flowchart of a molecular generation method according to one example embodiment of this disclosure. The method includes the following operations:

Operation 201: Sample initial hierarchical embeddings.

The initial hierarchical embeddings include an initial graph embedding of a molecular graph hierarchy, an initial subgraph embedding of a subgraph hierarchy, and an initial atomic embedding of an atomic hierarchy.

A molecular graph is composed of nodes and edges. The nodes represent atoms, and the edges represent chemical bonds connecting the atoms. A subgraph includes a regional molecular structure of the molecular graph.

The molecular graph may be expressed by a tuple G=(V, E), where V represents an atom set of the molecule, and E represents a chemical bond type set of the chemical bonds between the atoms. The atom set is V={v₁, . . . , vₙ}, which totally includes n atoms. The chemical bond type set is E={e₍ᵥ_{ᵢ}_{, v}_{ⱼ})|vᵢ, vⱼ∈V}, where e₍ᵥ_{ᵢ}_{, v}_{ⱼ}) represents a type of a chemical bond between atom vᵢ and atom vⱼ. After the atom set forming the molecule is determined, an atom type set X={x₁, . . . , xₙ} included in the molecule may be determined.

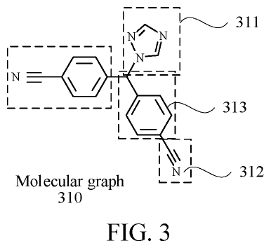

FIG. 3 shows a schematic diagram of hierarchies of a molecular structure according to one example embodiment of this disclosure. A molecular graph 310 is composed of atoms 312 and edges 313. The atoms 312 are connected to each other through the edges 313. The molecular graph 310 includes at least one subgraph 311, and the subgraph 311 includes a regional molecular structure of the molecular graph 310. As shown in FIG. 3, each dashed-line part represents a subgraph 311. A subgraph division manner shown in FIG. 3 is not a unique manner. Different subgraphs may be obtained by dividing a molecular graph in another manner.

In some embodiments, to generate a new molecule, a computer device obtains the initial graph embedding

$${z}_{t}^{X} ,$$

the initial subgraph embedding

$${z}_{t}^{M} ,$$

and the initial atomic embedding through sampling based on a standard Gaussian distribution, and the obtained initial hierarchical embeddings comply with the Gaussian distribution.

Operation 202: Denoise the initial hierarchical embeddings based on a hierarchical relationship among the molecular graph, the subgraph, and the atoms to obtain target hierarchical embeddings.

The target hierarchical embeddings include a target graph embedding of a molecular graph hierarchy, a target subgraph embedding of a subgraph hierarchy, and a target atomic embedding of an atomic hierarchy.

In the process of denoising the initial hierarchical embeddings, the computer device needs to consider a relationship among the atomic embedding, the subgraph embedding, and the molecular graph embedding. The atomic embedding, the subgraph embedding, and the molecular graph embedding are updated to predict to-be-removed noise, thus removing predicted noises from the initial hierarchical embeddings, to obtain the target hierarchical embeddings.

Operation 203: Decode the target hierarchical embeddings to obtain molecular structure information of a target molecule.

The molecular structure information includes atom types of atoms in the target molecule and chemical bond types of inter-atomic chemical bonds.

In some embodiments, in the process of decoding the target hierarchical embeddings, the computer device transforms the graph embedding into a series of subgraph segments by using an autoregressive model implemented by a single-layer recurrent neural network, and then predicts connections between these subgraph segments to construct the target molecule.

In some embodiments, the computer device decodes the target hierarchical embeddings to obtain an n×n×1 target molecular structure matrix to represent the molecular structure information. In the target molecular structure matrix, n represents n atoms, where data in dimension 1 represents a type of chemical bonds between the atoms. For example, values of the dimension may be 0, 1, and 2. Therefore, when a value of the type of the chemical bonds between a first atom and a second atom is 0, it indicates that no chemical bond exists between the first atom and the second atom. When the value is 1, it indicates that a single bond exists between the first atom and the second atom. When the value is 2, it may indicate that double bonds exist between the first atom and the second atom.

The embodiments of this disclosure include the initial graph embedding corresponding to the molecular graph hierarchy, the initial subgraph embedding corresponding to the subgraph hierarchy, and the initial atomic embedding corresponding to the atomic hierarchy, which are generated based on the molecular structure. In addition, the initial hierarchical embeddings are denoised based on the hierarchical relationship among the molecular graph, the subgraph, and the atoms. The target hierarchical embeddings are then decoded, thus obtaining the molecular structure information of the target molecule. The denoising is performed on different hierarchies of the molecular structure based on the hierarchical relationship among the molecular graph, the subgraph, and the atoms, thus more fully using the hierarchical structures inside the molecule and facilitating enhancement of diversity of the generated molecule. In addition, more fully using the hierarchical structures of the molecule for denoising is conducive to increasing an upper limit of the number of generated molecules and generating molecules with higher quality. In addition, the subgraph embedding is used as processing data of the machine learning model, so that the regional structure information of the molecule can be determined through the subgraph. This approach is conducive to lowering a storage requirement in a processing process of the machine learning model and reducing time consumption, thereby facilitating large-scale molecular generation.

In this embodiment of this disclosure, the initial hierarchical embeddings are denoised based on the idea of a diffusion model. The diffusion model is a generation model, and includes two Markov chains which are respectively a forward diffusion process and a reverse denoising process. The denoising on the initial hierarchical embeddings is the reverse denoising process.

In the forward diffusion process, for a data sample z₀~q(z₀), the forward diffusion process may be expressed by

$$q ( {z}_{1 : T} \vert {z}_{0} ) = {\prod }_{t = 1}^{T} q ( {z}_{t} \vert {z}_{t - 1} ) ,$$

where T represents a total time step; t represents a current time step; z represents the data sample; and q(z) represents a function with which the data sample complies.

In the forward diffusion process, a series of noise latent variables with gradually increased noises are generated by gradually adding Gaussian noise into the data sample, z_{1:T}=z₁, z₂, . . . , z_{T}. The forward diffusion process performed at the time step t may be expressed by q(zₜ|zₜ₋₁)= [symbol] (zₜ; \sqrt{1−βₜ}zₜ₋₁, βₜI), where N represents Gaussian noise; βₜ represents a hyper-parameter configured for controlling the quantity of Gaussian noise added into zₜ₋₁ at the time step t, βₜ∈(0,1), where βₜ is determined by noise scheduling, helping to ensure that a [symbol] final latent variable z_{T} approach standard Gaussian noise, i.e. z_{T}~ [symbol] (0, I).

The reverse denoising process at a time step may be expressed by q(zₜ₋₁|zₜ). Since it is hard to process q(zₜ₋₁₁|zₜ), q(zₜ₋₁zₜ) may be replaced at each time step through parameterized Gaussian transformation p_{θ}(zₜ₋₁|zₜ). The parameterized Gaussian transformation p_{θ}(zₜ₋₁|zₜ) is similar to q(zₜ₋₁|zₜ), where p_{θ}(zₜ₋₁|zₜ)= [symbol] (zₜ₋₁; μ_{θ}(zₜ, t), σ²I), μ_{θ} representing a neural network having a learnable parameter θ, and σ² representing a variance. In the reverse denoising process, a noise variable z_{T:1} can be removed through iteration. The denoising process can be expressed by

$${p}_{θ} ( {z}_{0 : T} ) = p ( {z}_{T} ) {\prod }_{t = 1}^{T} {p}_{θ} ( {z}_{t - 1} \vert {z}_{t} ) ,$$

where p(z_{T}) represents a standard Gaussian distribution.

In this embodiment of this disclosure, the denoising process on the initial hierarchical embeddings includes N denoising operations. First, the computer device performs a first denoising operation on the initial hierarchical embeddings based on the hierarchical relationship to obtain first intermediate hierarchical embeddings.

The process of performing the first denoising operation is the denoising performed by the computer device at a first time step in the reverse denoising process. The initial subgraph embedding, the initial graph embedding, and the initial atomic embedding that are included in the initial hierarchical embeddings comply with the standard Gaussian distribution.

Performing the first denoising operation on the initial hierarchical embeddings is that the computer device performs the first denoising operation on the initial graph embedding, the initial subgraph embedding, and the initial atomic embedding respectively, and the obtained first intermediate hierarchical embeddings include a first intermediate graph embedding, a first intermediate subgraph embedding, and a first intermediate atomic embedding.

After n denoising operations are performed in sequence, the computer device performs an (n+1)^{th} denoising operation on n^{th} intermediate hierarchical embeddings based on the hierarchical relationship, to obtain (n+1)^{th} intermediate hierarchical embeddings.

The n^{th} intermediate hierarchical embeddings are obtained by denoising (n−1)^{th} intermediate hierarchical embeddings. The n^{th} intermediate hierarchical embeddings include an n^{th} intermediate graph embedding of a molecular graph hierarchy, an n^{th} intermediate subgraph embedding of a subgraph hierarchy, and an n^{th} intermediate atomic embedding of an atomic hierarchy.

For example, in a case that n is 1, that is, when one instance of denoising has been performed, obtained latent variables are the first intermediate hierarchical embeddings. The computer device performs a second denoising operation on the first intermediate hierarchical embeddings to obtain third intermediate hierarchical embeddings. For another example, in a case that n is 5, that is, when five instances of denoising has been performed, obtained latent variables are fifth intermediate hierarchical embeddings. The computer device performs a sixth denoising operation on the fifth intermediate hierarchical embeddings to obtain sixth intermediate hierarchical embeddings.

After N−1 instances of denoising are performed in sequence, the computer device performs an n^{th} denoising operation on (N−1)^{th} intermediate hierarchical embeddings based on the hierarchical relationship to obtain target hierarchical embeddings.

In this embodiment of this disclosure, it is assumed that the denoising includes the N denoising operations, and after the n^{th} denoising operation is completed, the target hierarchical embeddings can be obtained. For example, N is 5. After five instances of denoising is performed on the initial hierarchical embeddings, the target hierarchical embeddings can be obtained.

In the process of each denoising operation, the computer device performs noise prediction based on the hierarchical relationship and intermediate hierarchical embeddings denoised at a previous time step, so as to obtain predicted noises of different hierarchies at a current time step; and denoises, based on the predicted noises, the intermediate hierarchical embeddings denoised at the previous time step, so as to obtain intermediate hierarchical embeddings denoised at the current time step.

For example, in the process of the first denoising operation, the computer device performs noise prediction based on the hierarchical relationship and initial hierarchical embeddings, to obtain first predicted noises of different hierarchies. The first predicted noises of different hierarchies include a first molecular graph predicted noise, a first subgraph predicted noise, and a first atomic predicted noise. Later, the initial hierarchical embeddings are denoised based on the first predicted noises of different hierarchies to obtain the first intermediate hierarchical embeddings.

For another example, in the process of the n^{th} denoising operation, the computer device performs noise prediction based on the hierarchical relationship and the (N−1)^{th} intermediate hierarchical embeddings, to obtain n^{th} predicted noises of different hierarchies. The n^{th} predicted noises of different hierarchies include a first molecular graph predicted noise, a first subgraph predicted noise, and a first atomic predicted noise. Later, the (N−1)^{th} intermediate hierarchical embeddings are denoised based on the n^{th} predicted noises of different hierarchies to obtain the N^{th} intermediate hierarchical embeddings.

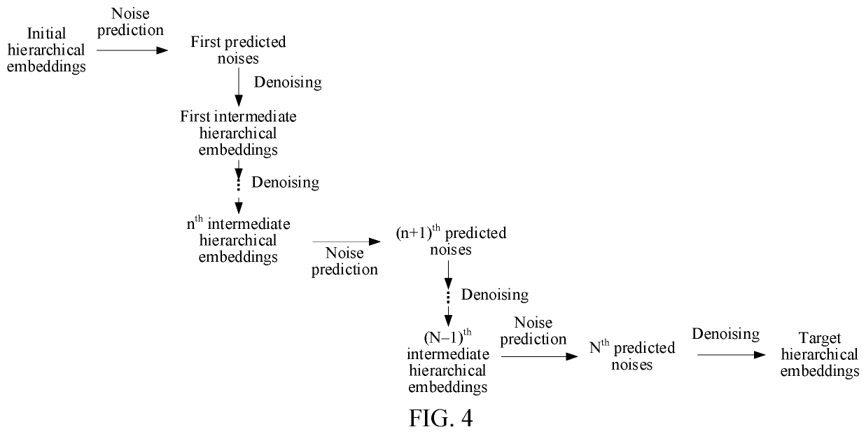

For still another example, FIG. 4 shows a schematic diagram of N denoising operations according to one example embodiment of this disclosure. The computer device performs noise prediction based on initial hierarchical embeddings to obtain first predicted noises of different hierarchies. Later, the initial hierarchical embeddings are denoised based on the first predicted noises to obtain first intermediate hierarchical embeddings. The computer device then performs noise prediction based on a hierarchical relationship and the first intermediate hierarchical embeddings to obtain second predicted noises of different hierarchies, and denoises the first intermediate hierarchical embeddings based on the second predicted noises to obtain second intermediate hierarchical embeddings. After n denoising operations are performed, n^{th} intermediate hierarchical embeddings are obtained. The computer device performs noise prediction based on the n^{th} intermediate hierarchical embeddings and the hierarchical relationship to obtain (n+1)^{th} predicted noises of different hierarchies, and denoises the n^{th} intermediate hierarchical embeddings based on the (n+1)^{th} predicted noises of different hierarchies to obtain (n+1)^{th} intermediate hierarchical embeddings. After (N−1) denoising operations are performed, the computer device performs noise prediction based on (N−1)^{th} intermediate hierarchical embeddings and the hierarchical relationship to obtain n^{th} predicted noises of different hierarchies, and denoises the n^{th} intermediate hierarchical embeddings based on the n^{th} predicted noises of different hierarchies to obtain generated hierarchical embeddings.

In an implementation, if initial hierarchical embeddings sampled in two molecule generation processes are the same, generated hierarchical embeddings obtained through N denoising operations in the two molecule generation processes are also the same, and the same molecular structure information may be finally obtained. Therefore, to increase diversity of structure information of generated molecules, the computer device may add additional information into the intermediate hierarchical embeddings after each denoising operation ends, to increase uncertainty. For example, the computer device may add Gaussian noise that is generated in any manner (for example, by sampling) into the intermediate hierarchical embeddings, and then continues to perform a next denoising operation based on noise-added intermediate hierarchical embeddings. In this way, even if the same initial hierarchical embeddings are used, different generated molecular structures may be generated in different denoising processes.

For example, n^{th} intermediate noises corresponding to different hierarchies may be obtained through sampling.

The computer device respectively acquires the n^{th} intermediate noises of the atomic hierarchy, the subgraph hierarchy, and the molecular graph hierarchy. The n^{th} intermediate noises corresponding to different hierarchies may be the same or different.

Noise addition is performed on the n^{th} intermediate hierarchical embeddings based on the n^{th} intermediate noises to obtain noise-added n^{th} intermediate hierarchical embeddings. Finally, noise prediction is performed based on the noise-added n^{th} intermediate hierarchical embeddings to obtain (n+1)^{th} predicted noises of different hierarchies.

The computer device performs noise addition on an n^{th} intermediate atomic embedding based on the n^{th} intermediate noise of the atomic hierarchy to obtain a noise-added n^{th} intermediate atomic embedding; performs noise addition on an n^{th} intermediate subgraph embedding based on the n^{th} intermediate noise of the subgraph hierarchy to obtain a noise-added n^{th} intermediate subgraph embedding; and performs noise addition on an n^{th} intermediate graph embedding based on the n^{th} intermediate noise of the molecular graph hierarchy to obtain a noise-added n^{th} intermediate graph embedding.

Since the noise addition is performed on the intermediate hierarchical embeddings obtained after each denoising operation, in a case that the same initial embedding vectors are sampled in a plurality of molecule generation processes, corresponding generated hierarchical embeddings are also different. Thus, generated molecules are diversified.

The following uses the (n+1)^{th} denoising operation as an example through one example embodiment to describe a specific process of each denoising operation.

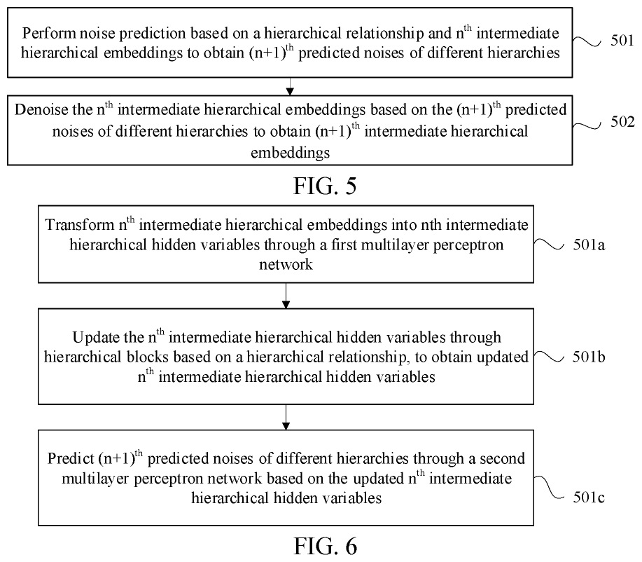

FIG. 5 shows a flowchart of a denoising process according to one example embodiment of this disclosure. The process includes the following operations.

Operation 501: Perform noise prediction based on a hierarchical relationship and n^{th} intermediate hierarchical embeddings to obtain (n+1)^{th} predicted noises of different hierarchies.

The (n+₁)^{th} predicted noises of different hierarchies include an (n+1)^{th} molecular graph predicted noise, an (n+1)^{th} subgraph predicted noise, and an (n+1)^{th} atomic predicted noise.

In the process of each denoising operation, since it is relatively difficult to directly predict denoised hierarchical embeddings, the computer device first performs the noise prediction and then removes the predicted noises from the n^{th} intermediate hierarchical embeddings. In this way, a denoising effect is enhanced by gradual denoising, thereby facilitating generation of generated molecules with higher quality and enhancing diversity of the generated molecules.

In an implementation example, the denoising is performed by a hierarchical denoising network. The hierarchical denoising network includes a first multilayer perceptron network, a second multilayer perceptron network, and at least two hierarchical blocks located between the first multilayer perceptron network and the second multilayer perceptron network.

FIG. 6 shows a flowchart of performing a denoising process by a hierarchical denoising network according to one example embodiment of this disclosure. The process includes the following operations.

Operation 501a: Transform the n^{th} intermediate hierarchical embeddings into n^{th} intermediate hierarchical hidden variables through the first multilayer perceptron network.

The n^{th} intermediate hierarchical hidden variables include an n^{th} intermediate graph hidden variable of a molecular graph hierarchy, an n^{th} intermediate subgraph hidden variable of a subgraph hierarchy, and an n^{th} intermediate atomic hidden variable of an atomic hierarchy.

In the hierarchical denoising network, first, the computer device transforms the hierarchical embeddings into hidden space variables corresponding to the hierarchical embeddings through the first multilayer perceptron network in the denoising network.

In some embodiments, three multilayer perceptrons (MLPs) exist in the first multilayer perceptron network. The three multilayer perceptrons are configured to respectively transform the intermediate hierarchical embeddings of different hierarchies. The n^{th} intermediate atomic embedding is transformed through an MLP to obtain an n^{th} intermediate atomic hidden variable

$${h}_{t}^{X} = MLP ( {z}_{t}^{X} ) .$$

The n^{th} intermediate subgraph embedding is transformed through an MLP to obtain an n^{th} intermediate subgraph hidden variable

$${h}_{t}^{M} = MLP ( {z}_{t}^{M} ) .$$

The n^{th} intermediate graph embedding is transformed through an MLP to obtain an n^{th} intermediate graph hidden variable

$${h}_{t}^{G} = MLP ( {z}_{t}^{G} ) .$$

Operation 501b: Update the n^{th} intermediate hierarchical hidden variables through the hierarchical blocks based on the hierarchical relationship, to obtain updated n^{th} intermediate hierarchical hidden variables.

In the process of updating the n^{th} intermediate hierarchical hidden variables through the hierarchical blocks, the computer device respectively updates the n^{th} intermediate atomic hidden variable, the n^{th} intermediate subgraph hidden variable, and the n^{th} intermediate graph hidden variable through the hierarchical blocks based on the hierarchical relationship, to obtain the updated n^{th} intermediate hierarchical hidden variables.

FIG. 7 shows a schematic diagram of a structure of a hierarchical block according to one example embodiment of this disclosure. The hierarchical block includes a third multilayer perceptron 701, a fourth multilayer perceptron 702, a first attention network 703, a fifth multilayer perceptron 704, and a neighborhood aggregator 705. The third multilayer perceptron 701 is configured to update the n^{th} intermediate atomic hidden variable. The fourth multilayer perceptron 702 and the first attention network 703 are configured to update the n^{th} intermediate subgraph hidden variable. The fifth multilayer perceptron 704 and the neighborhood aggregator 705 are configured to update the n^{th} intermediate graph hidden variable. X represents the n^{th} intermediate atomic hidden variable; M represents the n^{th} intermediate subgraph hidden variable; G represents the n^{th} intermediate graph hidden variable; X′ represents an updated n^{th} intermediate atomic hidden variable; M′ represents an updated n^{th} intermediate subgraph hidden variable; and G′ represents an updated n^{th} intermediate graph hidden variable.

The following respectively describes the update processes of the n^{th} intermediate atomic hidden variable, the n^{th} intermediate subgraph hidden variable, and the n^{th} intermediate graph hidden variable.

I. The n^{th} intermediate atomic hidden variable is updated based on the n^{th} intermediate atomic hidden variable and the n^{th} intermediate subgraph hidden variable to obtain the updated n^{th} intermediate atomic hidden variable.

In the process of updating the n^{th} intermediate atomic hidden variable, a relationship between the atomic embedding and the subgraph embedding needs to be considered.

First, the n^{th} intermediate atomic hidden variable is transformed through the third multilayer perceptron to obtain a first transformed hidden variable, and the n^{th} intermediate subgraph hidden variable is transformed through the third multilayer perceptron to obtain a second transformed hidden variable.

The first transformed variable obtained by transforming the n^{th} intermediate atomic hidden variable through the third multilayer perceptron is

$$MLP ( {h}_{t}^{X} ) ,$$

and similarly, the second transformed variable is

$$MLP ( {h}_{t}^{M} ) .$$

Later, the first transformed hidden variable is fused with the second transformed hidden variable to obtain a first fused variable. Finally, the first fused variable is transformed through the third multilayer perceptron to obtain the updated n^{th} intermediate atomic hidden variable. Thus, the finally obtained updated n^{th} intermediate atomic hidden variable is

$${h}_{t}^{{X}^{′}} = MLP [ MLP ( {h}_{t}^{X} ) + MLP ( {h}_{t}^{M} ) ] .$$

In some embodiments, the n^{th} intermediate atomic hidden variable and the n^{th} intermediate subgraph hidden variable are respectively transformed by using different third multilayer perceptrons. In the transformation process, the different third multilayer perceptrons have independent parameters, and parameters between the different third multilayer perceptrons are not shared.

II. The n^{th} intermediate subgraph hidden variable is updated based on the n^{th} intermediate subgraph hidden variable and the n^{th} intermediate atomic hidden variable to obtain the updated n^{th} intermediate subgraph hidden variable.

First, the computer device determines a first attention score through the first attention network based on the n^{th} intermediate atomic hidden variable and the n^{th} intermediate subgraph hidden variable. Moreover, the n^{th} intermediate atomic hidden variable is weighted based on the first attention score to obtain a weighted n^{th} intermediate atomic hidden variable.

In some embodiments, the computer device calculates first attention scores of the n^{th} intermediate atomic hidden variable and the n^{th} intermediate subgraph hidden variable by using dot products. Moreover, the first attention scores are multiplied by the n^{th} intermediate atomic hidden variable to implement the weighting on the n^{th} intermediate atomic hidden variable.

In some embodiments, there are a plurality of n^{th} intermediate atomic hidden variables and a plurality of n^{th} intermediate subgraph hidden variables. For an i^{th} intermediate subgraph hidden variable, its corresponding first attention score is a sum of dot products of the i^{th} intermediate subgraph hidden variable and the n^{th} intermediate atomic hidden variables, and the first attention score is Z softmax

$$( {h}_{t , i}^{M} {{h}_{t , j}^{X}}^{T} ) ,$$

and the weighted n^{th} intermediate atomic hidden variable is Σⱼ softmax

$$( {h}_{t , i}^{M} {{h}_{t , j}^{X}}^{T} ) MLP ( {h}_{t , j}^{X} ) ,$$

where j represents the number of n^{th} intermediate atomic hidden variables.

Then, the computer device transforms the n^{th} intermediate subgraph hidden variable through the fourth multilayer perceptron to obtain a third transformed hidden variable. The third transformed hidden variable obtained by transforming the n^{th} intermediate subgraph hidden variable through the fourth multilayer perceptron is

$$MLP ( {h}_{t , i}^{M} ) .$$

Later, the computer device fuses the third transformed hidden variable with the weighted n^{th} intermediate atomic hidden variable to obtain a second fused variable. Finally, the computer device transforms the second fused variable through the fourth multilayer perceptron to obtain the updated n^{th} intermediate subgraph hidden variable. Thus, the updated n^{th} intermediate subgraph hidden variable is

$${h}_{t , i}^{{M}^{′}} = MLP ( {\sum }_{j} softmax ( {h}_{t , i}^{M} {{h}_{t , j}^{X}}^{T} ) MLP ( {h}_{t , j}^{X} ) + MLP ( {h}_{t , i}^{M} ) ) .$$

In some embodiments, the second fused variable and the n^{th} intermediate subgraph hidden variable are transformed by using different fourth multilayer perceptrons. There are independent parameters in different transformation processes, and parameters are not shared in different transformations.

III. The n^{th} intermediate graph hidden variable is updated based on the n^{th} intermediate subgraph hidden variable, the n^{th} intermediate atomic hidden variable, and the n^{th} intermediate graph hidden variable to obtain the updated n^{th} intermediate graph hidden variable.

First, the computer device transforms the n^{th} intermediate graph hidden variable through the fifth multilayer perceptron to obtain a fourth transformed variable, and the obtained fourth transformed variable is

$$MLP ( {h}_{t}^{G} ) .$$

Then, neighborhood aggregation is performed on the n^{th} intermediate atomic hidden variable through the neighborhood aggregator to obtain a first aggregated variable. In addition, neighborhood aggregation is performed on the n^{th} intermediate subgraph hidden variable through the neighborhood aggregator to obtain a second aggregated variable.

In some embodiments, the n^{th} intermediate atomic hidden variable and the n^{th} intermediate subgraph hidden variable are processed through the neighborhood aggregation, and the intermediate graph embedding is updated based on neighborhood aggregation. Representations of the atomic hierarchy and the subgraph hierarchy are obtained through principal neighborhood aggregation pooling to update the intermediate hidden variable of the molecular graph hierarchy. After the neighborhood aggregation, the obtained first aggregated variable is

$$PNA ( {h}_{t}^{X} ) ,$$

and the second aggregated variable is

$$PNA ( {h}_{t}^{M} ) .$$

Later, the computer device fuses the fourth transformed variable, the first aggregated variable, and the second aggregated variable to obtain a third fused variable. Finally, the computer device transforms the third fused variable through the fifth multilayer perceptron to obtain the updated n^{th} intermediate graph hidden variable. The finally obtained updated n^{th} intermediate graph hidden variable is

$${h}_{t}^{{G}^{′}} = MLP ( PNA ( {h}_{t}^{X} ) + PNA ( {h}_{t}^{M} ) + MLP ( {h}_{t}^{G} ) ) .$$

In some embodiments, the third fused variable and the n^{th} intermediate graph hidden variable are transformed by using different fifth multilayer perceptrons. There are independent parameters in different transformation processes, and parameters are not shared in different transformations.

Operation 501c: Predict the (n+1)^{th} predicted noises of different hierarchies through the second multilayer perceptron network based on the updated n^{th} intermediate hierarchical hidden variables.

After the updated n^{th} intermediate hierarchical hidden variables are obtained, the computer device predicts a molecular graph hierarchical noise

$$MLP ( {h}_{t}^{{G}^{′}} ) ,$$

a subgraph hierarchical noise

$$MLP ( {h}_{t}^{{M}^{′}} ) ,$$

and an atomic hierarchical noise

$$MLP ( {h}_{t}^{{X}^{′}} )$$

from the n^{th} intermediate hierarchical hidden variables through the second multilayer perceptron network, and adds the predicted molecular graph hierarchical noise, subgraph hierarchical noise, and atomic hierarchical noise into the n^{th} intermediate hierarchical embeddings as noise residuals, so that an (n+1)^{th} molecular graph predicted noise of the molecular graph hierarchy is

$${\hat{ϵ}}_{t}^{G} = {z}_{t}^{G} + MLP ( {h}_{t}^{{G}^{′}} ) ,$$

an (n+1)^{th} subgraph predicted noise of the subgraph hierarchy is

$${\hat{ϵ}}_{t}^{M} = {z}_{t}^{M} + MLP ( {h}_{t}^{{M}^{′}} ) ,$$

and an (n+1)^{th} atomic predicted noise of the atomic hierarchy is

$${\hat{ϵ}}_{t}^{X} = {z}_{t}^{X} + MLP ( {h}_{t}^{{X}^{′}} ) .$$

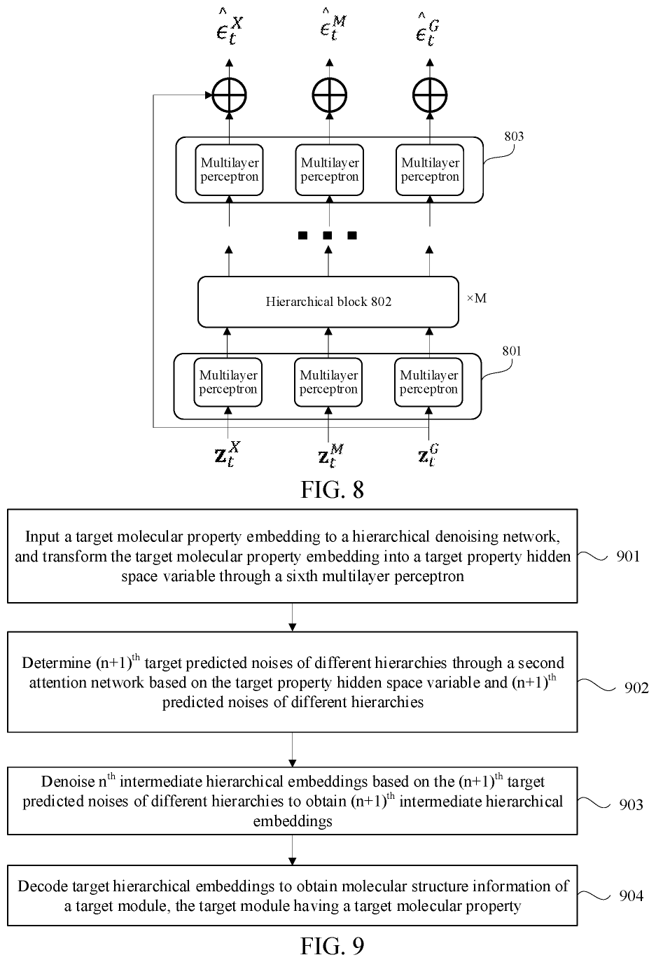

FIG. 8 shows a schematic diagram of a structure of a hierarchical denoising network according to one example embodiment of this disclosure. A first multilayer perceptron network 801 is included. The first multilayer perceptron network 801 includes three different multilayer perceptrons to respectively transform initial embeddings of different hierarchies.

$${z}_{t}^{X}$$

represents an atomic embedding;

$${z}_{t}^{M}$$

represents a subgraph embedding; and

$${z}_{t}^{G}$$

represents a graph embedding. An output end of the first multilayer perceptron network is connected to an input end of a hierarchical block 802. In some examples, the hierarchical denoising network includes M hierarchical blocks that are connected in series, and are configured for performing M instances of update on the intermediate hierarchical hidden variables. An output end of an m^{th} hierarchical block is connected to an input end of the second multilayer perceptron network 803. The second multilayer perceptron network 803 is configured for predicting noises and noise residuals of different hierarchies, and the denoising network adds the noise residuals of different hierarchies into the denoised initial hierarchical embeddings.

Operation 502: Denoise the n^{th} intermediate hierarchical embeddings based on the (n+1)^{th} predicted noises of different hierarchies to obtain (n+1)^{th} intermediate hierarchical embeddings.

The computer device denoises the n^{th} intermediate atomic embedding based on the (n+1)^{th} atomic predicted noise to obtain an (n+1)^{th} intermediate atomic embedding; denoises the n^{th} intermediate subgraph embedding based on the (n+1)^{th} subgraph predicted noise to obtain an (n+1)^{th} intermediate subgraph embedding; and denoises the n^{th} intermediate graph embedding based on the (n+1)^{th} molecular graph predicted noise to obtain an (n+1)^{th} intermediate graph embedding.

In some embodiments, for the process of denoising the n^{th} intermediate hierarchical embeddings based on the (n+1)^{th} predicted noises of different hierarchies, refer to a reverse denoising formula shown by the foregoing diffusion model.

In this embodiment of this disclosure, the N denoising operations are performed on the initial hierarchical embeddings to obtain the target hierarchical embeddings. During the denoising is performed based on the hierarchical relationship among the molecular graph, the subgraph, and the atoms, information interaction of the atoms, the subgraph, and the entire graph in a hidden space, so that relationships between different molecular feature hierarchies can be effectively caught, and it is conducive to more accurately generating diversified molecular structures. In addition, compared with performing a diffusion process on a chemical bond matrix, determining the regional structure information of the molecule through the subgraph embedding has a relatively low storage requirement and low time consumption.

In some implementation examples, a user has a demand for generating a molecule having specific molecular properties. Therefore, a conditional generation method can be used to input desired target molecular properties to the denoising network, thus generating molecular structure information with the target molecular properties.

The following describes the process of generating the molecular structure information based on the specific molecular properties through one example embodiment.

FIG. 9 shows a flowchart of a process of generating molecular structure information according to one example embodiment of this disclosure. The process includes the following operations.

Operation 901: Input a target molecular property embedding to the hierarchical denoising network, and transform the target molecular property embedding into a target property hidden space variable through the sixth multilayer perceptron.

It is assumed that s={s₁, s₂, . . . } represents the target molecular property embedding, where each dimension represents a specific molecular property, such as a molecular water solubility and a high syntheticity.

The computer device processes the target molecular property embedding through the sixth multilayer perceptron, and the obtained target property hidden space variable is MLP(s).

Operation 902: Determine (n+1)^{th} target predicted noises of different hierarchies through a second attention network (i.e. the property attention network) based on the target property hidden space variable and the (n+1)^{th} predicted noises of different hierarchies.

First, the computer device determines second attention scores of the target property hidden space variable and the (n+1)^{th} predicted noises of different hierarchies through the second attention network.

The second attention scores represent associations between the (n+1)^{th} predicted noises and the target property hidden space variable.

In a case of obtaining the molecular property embedding, the computer device can obtain queries

$${Q}^{X} = MLP ( {h}_{t}^{X} ) , {Q}^{M} = MLP ( {h}_{t}^{M} ) , and {Q}^{G} = MLP ( {h}_{t}^{G} )$$

of different hierarchies after processing the n^{th} intermediate atomic embedding, the n^{th} intermediate subgraph embedding, the n^{th} intermediate graph embedding, and the target molecular property embedding through the MLPs, and can determine a key K=MLP(s) and a value V=MLP(s).

In the process of updating the embeddings by using a cross-attention mechanism. First, the second attention scores are determined through dot products between the queries of different hierarchies and the key, and the attention scores are normalized through a softmax function, so that the second attention score of the atomic hierarchy is

$${α}_{ij}^{X} = softmax ( {Q}_{i}^{X} {K}_{j}^{T} / \sqrt{{d}_{k}} ) ,$$

the second attention score of the subgraph hierarchy is

$${α}_{ij}^{M} = softmax ( {Q}_{i}^{M} {K}_{j}^{T} / \sqrt{{d}_{k}} ) ,$$

and the second attention score of the molecular graph hierarchy is

$${α}_{j}^{G} = softmax ( {Q}^{G} {K}_{j}^{T} / \sqrt{{d}_{k}} ) ,$$

wherein dₖ represents a dimension of the key.

Later, the target property hidden space variable is weighted based on the second attention scores of the different hierarchies to obtain the (n+1)^{th} target predicted noises of the different hierarchies.

After obtaining the second attention scores, the computer device performs weighting calculation on the value V=MLP(s) based on the attention scores to obtain the updated intermediate hierarchical hidden variables. The updated atomic hierarchical hidden variable is

$${h}_{t}^{′X} = {\sum }_{j} {α}_{ij}^{X} {V}_{j ,}$$

the updated subgraph hierarchical hidden variable is

$${h}_{t}^{′M} = {\sum }_{j} {α}_{ij}^{M} {V}_{j} ,$$

and the updated graph hierarchical hidden variable is

$${h}_{t}^{′G} = {\sum }_{j} {α}_{j}^{G} {V}_{{j}^{′}} .$$

The updated intermediate hierarchical hidden variables include information of the target molecular property. It is conducive to guiding molecular generation in a direction of the target molecule in a direction of generating the target molecule with the specific molecular properties.

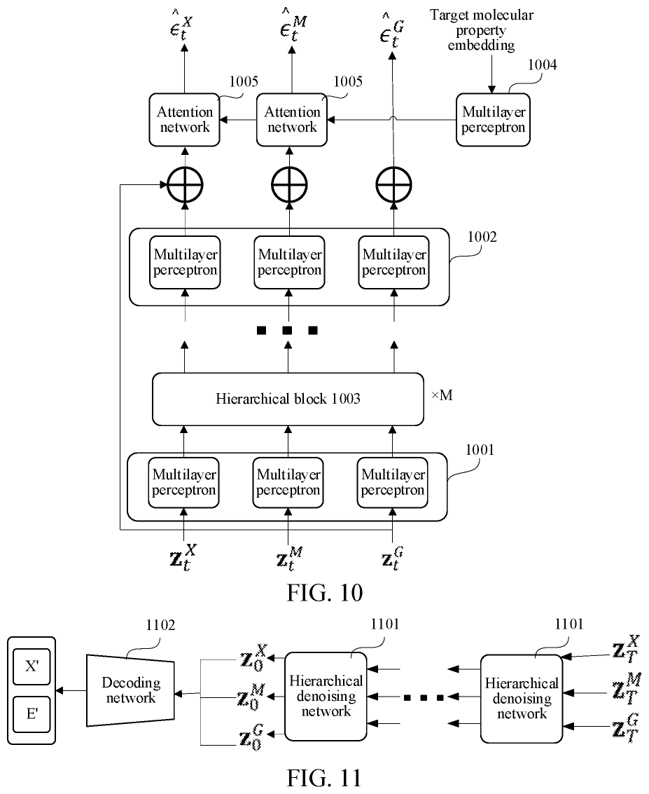

FIG. 10 shows a schematic diagram of a structure of a hierarchical denoising network according to another example embodiment of this disclosure. The hierarchical denoising network includes a first multilayer perceptron network 1001, a second multilayer perceptron network 1002, M hierarchical blocks 1003, a sixth multilayer perceptron 1004, and a second attention network 1005. The first multilayer perceptron network 1001 is configured for transforming the n^{th} intermediate hierarchical embeddings into n^{th} intermediate hierarchical hidden variables, where

$${Z}_{t}^{X}$$

represents an atomic embedding,

$${Z}_{t}^{M}$$

represents a subgraph embedding, and

$${Z}_{t}^{G}$$

represents a graph embedding. The hierarchical blocks 1003 are configured for updating the n^{th} intermediate hierarchical hidden variables, and the second multilayer perceptron network 1002 is configured for determining the (n+1)^{th} predicted noises of different hierarchies. The sixth multilayer perceptron 1004 is configured for transforming the target molecular property embedding into the target property hidden space variable. The second attention network 1005 is configured for determining the (n+1)^{th} target predicted noises of different hierarchies as

$${\hat{ϵ}}_{t}^{X} , {\hat{ϵ}}_{t}^{M} , and {\hat{ϵ}}_{t}^{G}$$

respectively based on the target property hidden space variable and the (n+1)^{th} predicted noises of different hierarchies.

Operation 903: Denoise the n^{th} intermediate hierarchical embeddings based on the (n+1)^{th} target predicted noises of different hierarchies to obtain (n+1)^{th} intermediate hierarchical embeddings.

For the process of denoising the n^{th} intermediate hierarchical embeddings based on the (n+1)^{th} target predicted noises of different hierarchies, refer to the process of denoising the n^{th} intermediate hierarchical embeddings based on the (n+1)^{th} predicted noises of different hierarchies in the foregoing embodiment, and details are not described again in this disclosure.

Operation 904: Decode target hierarchical embeddings to obtain molecular structure information of a target molecule, the target molecule having a target molecular property.

For a specific implementation process of this operation, refer to foregoing operation 203. Details are not described herein in this embodiment.

In this embodiment of this disclosure, by using a conditional generation mechanism, the intermediate hierarchical embeddings are denoised through the denoising network based on the target molecular property embedding, and the target molecular property and the hierarchical embeddings are combined to generate modules meeting a feature requirement. The modules are highly flexible, and the properties and molecular structures of the generated molecules can be flexibly controlled.

In an implementation example, after the target hierarchical embeddings are obtained, the target hierarchical embeddings are decoded through the decoding network, to obtain the molecular structure information of the target molecule.

In the decoding process, first, a target graph embedding feature, a target subgraph embedding feature, and a target atomic embedding feature are decoded through the decoding network, and the hierarchical embeddings are transformed into a series of subgraph segments. The subgraph segments include atoms and chemical bonds. Finally, connections between the subgraph segments are predicted through the decoding network in a link prediction manner, thus obtaining the molecular structure information.

In some embodiments, the foregoing decoding process may be implemented through a principal subgraph-variational auto-encoder (PS-VAI) model. The decoding network transforms the graph embedding into a series of subgraph segments by using an autoregressive model implemented by a single-layer recurrent neural network.

Schematically, FIG. 11 shows a schematic diagram of a structure of a molecular generation model according to one example embodiment of this disclosure. The molecular generation model includes a hierarchical denoising network 1101 and a decoder 1102. The computer device inputs the initial hierarchical embeddings (including the initial atomic embedding

$${z}_{T}^{X} ,$$

the initial subgraph embedding

$${z}_{T}^{M} ,$$

and the initial molecular graph embedding

$${z}_{T}^{G} )$$

to the denoising network, performs denoising through the hierarchical denoising network 1101 based on the hierarchical relationship to obtain the target hierarchical embeddings (including the target atomic embedding

$${z}_{0}^{X} ,$$

the target subgraph embedding

$${z}_{0}^{M} ,$$

and the target graph embedding

$${z}_{0}^{G} ) ,$$

and inputs the target hierarchical embeddings to the decoding network 1102 to obtain structure information of the target molecule, which includes atom types X′ of atoms in the target molecule and chemical bond types E′ of inter-atom chemical bonds.

Before the molecular structure information of the target molecule is generated through the foregoing method based on the initial hierarchical embeddings, the hierarchical denoising network and the decoding network need to be trained.

In a training process, training needs to be performed based on sample molecular structure information provided by a user, so that the decoding network learns the capability of generating the molecular structure information of the target molecule based on the target hierarchical embeddings, and the hierarchical denoising network can better use the features of hierarchical structures of the molecule to perform a reverse diffusion operation on different hierarchies of the molecule.

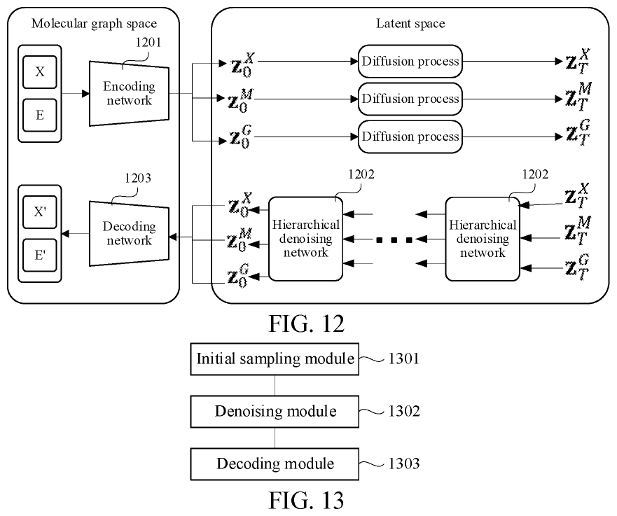

Schematically, FIG. 12 shows a schematic diagram of a structure of a molecular generation model in a training process according to one example embodiment of this disclosure. The molecular generation model includes an encoding network 1201, a hierarchical denoising network 1202, and a decoding network 1203. In an application process, the decoding network 1203 is configured for decoding the target hierarchical embeddings to obtain the molecular structure information of the target molecule, and the hierarchical denoising network 1202 is configured for denoising the initial hierarchical embeddings. In a training process, the encoding network 1201 is configured for encoding the sample molecular structure information to obtain sample hierarchical embeddings. The sample structure information belongs to a molecular graph space, and the sample hierarchical embeddings belong to a latent space. The decoding network 1203 is configured for decoding the sample hierarchical embeddings to obtain an estimated molecular structure information of the molecular graph space. The hierarchical denoising network 1202 is configured for denoising the sample noisy hierarchical embeddings.

Based on the molecular generation model shown in FIG. 12, in an implementation example, the molecular generation model needs to be trained in two stages. The following respectively describes training processes of the two stages.

In a first stage, the computer device trains the encoding network and the decoding network based on first sample molecular structure information of a first sample molecule.

First, the computer device inputs the first sample molecular structure information to the encoding network to obtain the first sample hierarchical embeddings outputted by the encoding network.

The first sample hierarchical embeddings include a first sample graph embedding of the molecular graph hierarchy, a first sample subgraph embedding of the subgraph hierarchy, and a first sample atomic embedding of the atomic hierarchy. The first sample molecular structure information includes an atom type X and a chemical bond type E.

The encoding network may be represented by [symbol] . An encoding formula of the encoding network may be q_{φ}(z^{X}, z^{M}, z^{G}|X, E)= [symbol] ( [symbol] (X, E), σI), where φ represents a trainable parameter of an encoder; z^{X} represents the first sample atomic embedding; z^{M} represents the first sample subgraph embedding; and z^{G} represents the first sample graph embedding.

Then, the first sample hierarchical embeddings are decoded through the decoding network to obtain estimated molecular structure information of an estimated sample molecule.

The decoding network may be represented by [symbol] . A decoding formula of the decoding network may be p_{ψ}(X, E|z^{X}, z^{M}, z^{G})=

$${\prod }_{i = 1}^{n} {p}_{ψ} ( {x}_{i} \vert {z}^{X} , {z}^{M} , {z}^{G} ) {\prod }_{i = 1}^{n} {\prod }_{j = 1 , j \neq i}^{n} {p}_{ψ} ( {e}_{( {v}_{i} , {v}_{j} )} \vert {z}^{X} , {z}^{M} , {z}^{G} ) ,$$

where ψ represents a trainable parameter of a decoder.

In some embodiments, an encoder and a decoder in the a PS-VAI are respectively used as the encoding network and a decoding network.

Later, an estimated loss of the decoding network is determined.

In some embodiments, a first reconstruction loss is determined based on a difference between the estimated molecular structure information and the first sample molecular structure information. In addition, a Kullback-Leibler divergence loss is determined based on a difference between the first sample hierarchical embeddings and a standard Gaussian distribution. Finally, a sum of the first reconstruction loss and the Kullback-Leibler divergence loss is determined as a total estimated loss.

Since the atom type X and the chemical bond type E are discrete features, a cross-entropy loss can be used as a reconstruction loss, and the reconstruction loss is L_{rec}=− [symbol] _{q}_{_{φ}}_{(z}_{^{X}}_{, z}_{^{M}}_{, z}_{^{G}}_{|X,E)}p_{ψ}(X, E|z^{X}, z^{M}, z^{G}). Furthermore, the Kullback-Leibler divergence loss is configured for training, so that a hidden space embedding is aligned with the standard Gaussian distribution p(z^{X}, z^{M}, z^{G}). The Kullback-Leibler divergence loss is [symbol] _{KL}=D_{KL}(q_{φ}(z^{X}, Z^{M}, z^{G}|X, E)∥p(z^{X}, Z^{M}, z^{G})). It can be determined that the total estimated loss is:

$$ℒ = {ℒ}_{rec} + {γℒ}_{KL} = - {𝔼}_{{q}_{ϕ} ( {z}^{X} , {z}^{M} , {z}^{G} \vert X , E )} {p}_{ψ} ( X , E \vert {z}^{X} , {z}^{M} , {z}^{G} ) + γ {D}_{KL} ( {q}_{ϕ} ( {z}^{X} , {z}^{M} , {z}^{G} \vert X , E ) \vert \vert p ( {z}^{X} , {z}^{M} , {z}^{G} ) )$$

where γ represents a hyper-parameter for controlling a weight of the Kullback-Leibler Kullback-Leibler divergence loss. The Kullback-Leibler divergence loss and the estimated loss are used as the total estimated loss, thus balancing a reconstruction error and KL divergence between a prior distribution and a posterior distribution of the hierarchical embeddings.

Finally, the encoding network and the decoding network are trained based on the total estimated loss.

The process of training the encoding network and the decoding network based on the total estimated loss is a process of continuously optimizing the encoding network and the decoding network to cause the trainable parameters φ and ψ to converge.

In a second stage, the hierarchical denoising network and the encoding network are trained based on second sample molecular structure information of a second sample molecule in a case that the training on the encoding network and the decoding network is completed.

In the second stage of training, the encoding network and the decoding network that have been trained and the untrained hierarchical denoising network are configured to form the molecular generation model, and the second sample molecular structure information is used as a sample. By determining a loss for noise deviations estimated in the noise addition process and by the denoising network, the hierarchical denoising network is trained. In addition, to increase an upper limit of optimization of the denoising network, the encoding network is also optimized in the second stage of training.

First, the computer device inputs the second sample molecular structure information to the encoding network to obtain second sample hierarchical embeddings outputted by the encoding network.

The second sample hierarchical embeddings include a second sample graph embedding of the molecular graph hierarchy, a second sample subgraph embedding of the subgraph hierarchy, and a second sample atomic embedding of the atomic hierarchy.

For the process of encoding the second sample structure information through the encoding network, refer to the process of encoding the first sample structure information through the encoding network in the foregoing first stage. Details are not described herein again in this embodiment.

Then, the computer device samples sample noises of different hierarchies. Moreover, noise addition is performed on the second sample hierarchical embeddings based on the sample noises of different hierarchies, to obtain second sample noisy hierarchical embeddings.

The second sample noisy hierarchical embeddings include a second sample noisy graph embedding of the molecular graph hierarchy, a second sample noisy subgraph embedding of the subgraph hierarchy, and a second sample noisy atomic embedding of the atomic hierarchy.

For the process of performing noise addition on the second sample hierarchical embeddings, refer to the forward diffusion process of the diffusion model in the foregoing embodiment. Details are not described herein again in this embodiment.

Then, noise prediction is performed through the hierarchical denoising network based on the second sample noisy hierarchical embeddings to obtain predicted noises of different hierarchies. In addition, a noise prediction loss is determined based on a difference between the predicted noises of different hierarchies and the sample noises of different hierarchies.

In the denoising process, the computer device completes a T-operation reverse denoising process by iterating time steps T. After the iteration of the denoising network is completed, the predicted noises can be obtained:

$${ϵ}_{θ} ( {Z}_{t}^{X} , {Z}_{t}^{M} , {Z}_{t}^{G} , t ) .$$

In some embodiments, an expected square error between the predicted noises and the sample noises can be determined as an estimated noise loss. The estimated noise loss is:

$$L = {E}_{{Z}_{0} , ϵ ∼ N ( 0 , I ) , t} [ w ( t ) {‖ ϵ - {ϵ}_{θ} ( {Z}_{t}^{X} , {Z}_{t}^{M} , {Z}_{t}^{G} , t ) ‖}^{2} ]$$

where w(t) is a weighting item of a weight.

Finally, the hierarchical denoising network and the encoding network are trained based on the noise prediction loss.

The process of training the denoising network and the encoding network is a process of optimizing the denoising network and the encoding network to cause the estimated noise loss to converge.

In an implementation example, if the molecular generation model on which training is completed can generate the molecular structure information with a desired molecular property, in the training process, a sample molecular property also needs to be inputted to the hierarchical denoising network, so that the computer device trains the hierarchical denoising network and the encoding network based on the second molecular structure information of the second sample molecule and the sample molecular property.

In some embodiments, in the process of training the hierarchical denoising network based on the second sample structure information (also referred to as second sample molecular structure information in some examples) of the second sample molecule, the second sample molecular structure information is first inputted to the encoding network to obtain the second sample hierarchical embeddings outputted by the encoding network. The second sample noisy hierarchical embeddings include a second sample noisy graph embedding of the molecular graph hierarchy, a second sample noisy subgraph embedding of the subgraph hierarchy, and a second sample noisy atomic embedding of the atomic hierarchy. Then, noise prediction is performed through the hierarchical denoising network based on the second sample noisy hierarchical embeddings and a sample molecular property embedding corresponding to the sample molecular property, to obtain predicted noises of different hierarchies. In addition, a noise prediction loss is determined based on a difference between the predicted noises of different hierarchies and the sample noises of different hierarchies, and the hierarchical denoising network and the encoding network are trained based on the noise prediction loss. The noise prediction loss in the training process is:

$$L = {E}_{{Z}_{0} , ϵ ∼ N ( 0 , I ) , t} [ w ( t ) {‖ ϵ - {ϵ}_{θ} ( {Z}_{t}^{X} , {Z}_{t}^{M} , {Z}_{t}^{G} , t , s ) ‖}^{2} ]$$

In the embodiments of this disclosure, by training the hierarchical denoising network and the encoding and decoding networks, the hierarchical denoising network and the decoding network that have been trained can better use features of a hierarchical structure inside a molecule in a molecule generation process, thereby generating more diversified molecules with higher quality. In addition, parallel computing is performed through the embeddings of the graph hierarchy, the subgraph hierarchy, and the atomic hierarchy, so that a large-scale molecular generation task can be processed within relatively short time, and an efficient computing capability is achieved. This facilitates processing of a more complex molecular system and a larger-scale molecular generation task.

FIG. 13 shows a block diagram of a structure of a molecular generation apparatus according to one example embodiment of this disclosure. As shown in FIG. 13, the apparatus includes the following modules.
an initial sampling module 1301 configured to generate initial hierarchical embeddings, for example, obtaining the initial hierarchical embeddings through sampling, the initial hierarchical embeddings including an initial graph embedding of a molecular graph hierarchy, an initial subgraph embedding of a subgraph hierarchy, and an initial atomic embedding of an atomic hierarchy, a molecular graph being composed of nodes and edges, the nodes representing atoms, the edges representing chemical bonds connecting the atoms, and a subgraph including a regional molecular structure of the molecular graph;
a denoising module 1302 configured to denoise the initial hierarchical embeddings based on a hierarchical relationship among the molecular graph, the subgraph, and the atoms to obtain target hierarchical embeddings, the target hierarchical embeddings including a target graph embedding of the molecular graph hierarchy, a target subgraph embedding of the subgraph hierarchy, and a target atomic embedding of the atomic hierarchy; and
a decoding module 1303 is configured to decode the target hierarchical embeddings to obtain molecular structure information of a target molecule, the molecular structure information including atom types of the atoms in the target molecule and chemical bond types of inter-atomic chemical bonds.

In some embodiments, the denoising includes N denoising operations, N being a positive integer.

The denoising module 1302 is configured to:
perform a first denoising operation on the initial hierarchical embeddings based on the hierarchical relationship to obtain first intermediate hierarchical embeddings;
perform an (n+1)^{th} denoising operation on n^{th} intermediate hierarchical embeddings based on the hierarchical relationship to obtain (n+1)^{th} intermediate hierarchical embeddings, the n^{th} intermediate hierarchical embeddings including an n^{th} intermediate graph embedding of the molecular graph hierarchy, an n^{th} intermediate subgraph embedding of the subgraph hierarchy, and an n^{th} intermediate atomic embedding of the atomic hierarchy; and
perform an n^{th} denoising operation on (N−1)^{th} intermediate hierarchical embeddings based on the hierarchical relationship to obtain generated hierarchical embeddings.

In some embodiments, the denoising module 1302 is configured to:
perform noise prediction based on the hierarchical relationship and the n^{th} intermediate hierarchical embeddings to obtain (n+1)^{th} predicted noises of different hierarchies, the (n+1)^{th} predicted noises of different hierarchies include an (n+1)^{th} molecular graph predicted noise, an (n+1)^{th} subgraph predicted noise, and an (n+1)^{th} atomic predicted noise; and
denoise the n^{th} intermediate hierarchical embeddings based on the (n+1)^{th} predicted noises of different hierarchies to obtain the (n+1)^{th} intermediate hierarchical embeddings.

In some embodiments, the denoising is performed by a hierarchical denoising network. The hierarchical denoising network includes a first multilayer perceptron network, a second multilayer perceptron network, and at least two hierarchical blocks located between the first multilayer perceptron network and the second multilayer perceptron network.

The denoising module 1302 is configured to:
transform the n^{th} intermediate hierarchical embeddings into n^{th} intermediate hierarchical hidden variables through the first multilayer perceptron network, the n^{th} intermediate hierarchical hidden variables including an n^{th} intermediate graph hidden variable of the molecular graph hierarchy, an n^{th} intermediate subgraph hidden variable of the subgraph hierarchy, and an n^{th} intermediate atomic hidden variable of the atomic layer;
update the n^{th} intermediate hierarchical hidden variables through the hierarchical blocks based on the hierarchical relationship, to obtain updated n^{th} intermediate hierarchical hidden variables; and
predict the (n+1)^{th} predicted noises of different hierarchies through the second multilayer perceptron network based on the updated n^{th} intermediate hierarchical hidden variables.

In some embodiments, the denoising module 1302 is configured to:
update the n^{th} intermediate atomic hidden variable based on the n^{th} intermediate atomic hidden variable and the n^{th} intermediate subgraph hidden variable to obtain an updated n^{th} intermediate atomic hidden variable;
update the n^{th} intermediate subgraph hidden variable based on the n^{th} intermediate subgraph hidden variable and the n^{th} intermediate atomic hidden variable to obtain an updated n^{th} intermediate subgraph hidden variable; and
update the n^{th} intermediate graph hidden variable based on the n^{th} intermediate subgraph hidden variable, the n^{th} intermediate atomic hidden variable, and the n^{th} intermediate graph hidden variable to obtain an updated n^{th} intermediate graph hidden variable.

In some embodiments, each hierarchical block includes a third multilayer perceptron.

The denoising module 1302 is configured to:
transform, through the third multilayer perceptron, the n^{th} intermediate atomic hidden variable to obtain a first transformed hidden variable, and transform, through the third multilayer perceptron, the n^{th} intermediate subgraph hidden variable to obtain a second transformed hidden variable;
fuse the first transformed hidden variable with the second transformed hidden variable to obtain a first fused variable; and
transform the first fused variable through the third multilayer perceptron to obtain the updated n^{th} intermediate atomic hidden variable.

In some embodiments, each hierarchical block includes a fourth multilayer perceptron and a first attention network.

The denoising module 1302 is configured to:
determine a first attention score through the first attention network based on the n^{th} intermediate atomic hidden variable and the n^{th} intermediate subgraph hidden variable;
weight, based on the first attention score, the n^{th} intermediate atomic hidden variable to obtain a weighted n^{th} intermediate atomic hidden variable;
transform, through the fourth multilayer perceptron, the n^{th} intermediate subgraph hidden variable to obtain a third transformed hidden variable;
fuse the third transformed hidden variable with the weighted n^{th} intermediate atomic hidden variable to obtain a second fused variable; and
transform the second fused variable through the fourth multilayer perceptron to obtain the updated n^{th} intermediate subgraph hidden variable.

In some embodiments, each hierarchical block includes a fifth multilayer perceptron and a neighborhood aggregator.

The denoising module 1302 is configured to:
transform, through the fifth multilayer perceptron, the n^{th} intermediate graph hidden variable to obtain a fourth transformed variable;
perform, through the neighborhood aggregator, neighborhood aggregation on the n^{th} intermediate atomic hidden variable to obtain a first aggregated variable;
perform, through the neighborhood aggregator, neighborhood aggregation on the n^{th} intermediate subgraph hidden variable to obtain a second aggregated variable;
fuse the fourth transformed variable, the first aggregated variable, and the second aggregated variable to obtain a third fused variable; and

transform the third fused variable through the fifth multilayer perceptron to obtain the updated n^{th} intermediate molecular graph hidden variable.

In some embodiments, the hierarchical denoising network further includes a sixth multilayer perceptron and a second attention network.

The denoising module 1302 is further configured to:
input a target molecular property embedding to the hierarchical denoising network, and transform the target molecular property embedding into a target property hidden space variable through the sixth multilayer perceptron;
determine (n+1)^{th} target predicted noises of different hierarchies through the second attention network based on the target property hidden space variable and the (n+1)^{th} predicted noises of different hierarchies; and
denoise the n^{th} intermediate hierarchical embeddings based on the (n+1)^{th} target predicted noises of different hierarchies to obtain the (n+1)^{th} intermediate hierarchical embeddings.

In some embodiments, the denoising module 1302 is configured to:
determine, through the second attention network, second attention scores of the target property hidden space variable and the (n+1)^{th} predicted noises of different hierarchies, the second attention scores being configured for representing associations between the (n+1)^{th} predicted noises and the target property hidden space variable; and
weight the target property hidden space variable based on the second attention scores of the different hierarchies to obtain the (n+1)^{th} target predicted noises of the different hierarchies.

In some embodiments, the apparatus further includes:
a noise addition module configured to sample n^{th} intermediate noises corresponding to different hierarchies.

The noise addition module is further configured to perform noise addition on the n^{th} intermediate hierarchical embeddings based on the n^{th} intermediate noises to obtain noise-added n^{th} intermediate hierarchical embeddings.

The denoising module 1302 is configured to:

perform noise prediction based on the noise-added n^{th} intermediate hierarchical embeddings to obtain the (n+1)^{th} predicted noises of different hierarchies.

In some embodiments, the apparatus further includes:
a first training module configured to train an encoding network and a decoding network based on first sample molecular structure information of a first sample molecule, the encoding network being configured for encoding sample molecular structure information to obtain sample hierarchical embeddings, and the decoding network being configured for decoding the target hierarchical embeddings to obtain the molecular structure information of the target molecule; and
a second training module configured to train the hierarchical denoising network and the encoding network based on second sample molecular structure information of a second sample molecule in a case that the training on the encoding network and the decoding network is completed, the hierarchical denoising network being configured for denoising the initial hierarchical embeddings.

In some embodiments, the first training module is configured to:
input the first sample molecular structure information to the encoding network to obtain first sample hierarchical embeddings outputted by the encoding network, the first sample hierarchical embeddings including a first sample graph embedding of a molecular graph hierarchy, a first sample subgraph embedding of a subgraph hierarchy, and a first sample atomic embedding of an atomic hierarchy;
decode the first sample hierarchical embeddings through the decoding network to obtain estimated molecular structure information of an estimated sample molecule;
determine a first reconstruction loss based on a difference between the estimated molecular structure information and the first sample molecular structure information;
determine a Kullback-Leibler divergence loss based on a difference between the first sample hierarchical embeddings and a standard Gaussian distribution;
determine a sum of the first reconstruction loss and the Kullback-Leibler divergence loss as a total estimated loss; and
train the encoding network and the decoding network based on the total estimated loss.

In some embodiments, the second training module is configured to:
input the second sample molecular structure information to the encoding network to obtain second sample hierarchical embeddings outputted by the encoding network, the second sample hierarchical embeddings including a second sample graph embedding of a molecular graph hierarchy, a second sample subgraph embedding of a subgraph hierarchy, and a second sample atomic embedding of an atomic hierarchy;
sample sample noises of different hierarchies;
performing noise addition on the second sample hierarchical embeddings based on the sample noises of different hierarchies to obtain second sample noisy hierarchical embeddings, the second sample noisy hierarchical embeddings including a second sample noisy graph embedding of the molecular graph hierarchy, a second sample noisy subgraph embedding of the subgraph hierarchy, and a second sample noisy atomic embedding of the atomic hierarchy;
perform noise prediction through the hierarchical denoising network based on the second sample noisy hierarchical embeddings to obtain predicted noises of different hierarchies;
determine a noise prediction loss based on a difference between the predicted noises of different hierarchies the sample noises of different hierarchies; and
train the hierarchical denoising network and the encoding network based on the noise prediction loss.

In some embodiments, the second training module is configured to train the hierarchical denoising network and the encoding network based on the second molecular structure information and a sample molecular property of the second sample molecule.

In conclusion, in the embodiments of this disclosure, the initial graph embedding corresponding to the molecular graph hierarchy, the initial subgraph embedding corresponding to the subgraph hierarchy, and the initial atomic embedding corresponding to the atomic hierarchy are sampled based on the molecular structure. In addition, the initial hierarchical embeddings are denoised based on the hierarchical relationship among the molecular graph, the subgraph, and the atoms to obtain generated hierarchical embeddings. Finally, the generated hierarchical embeddings are then decoded, thus obtaining the molecular structure information of the generated molecule. The denoising is performed on different hierarchies of the molecular structure based on the hierarchical relationship among the molecular graph, the subgraph, and the atoms, thus more fully using the hierarchical structures inside the molecule and facilitating enhancement of diversity of the generated molecule. In addition, more fully using the hierarchical structures of the molecule for denoising is conducive to increasing the quantity generated molecules and generating generated molecules with higher quality. In addition, the subgraph embedding is configured for diffusion, so that the regional structure information of the molecule can be determined through the subgraph. It is conducive to lowering a storage requirement in the diffusion process and reducing time consumption, thereby facilitating large-scale molecular generation.

The apparatus provided in the foregoing embodiments is described using the division of the foregoing functional modules as an example. During actual application, the foregoing functions may be allocated to and completed by different functional modules according to requirements. In other words, an internal structure of the apparatus is divided into different functional modules, to complete all or part of the functions described above. In addition, the apparatus provided in the foregoing embodiments and the method embodiments fall within the same conception. For details of an implementation process of the apparatus, refer to the method embodiments. Details are not described herein again.

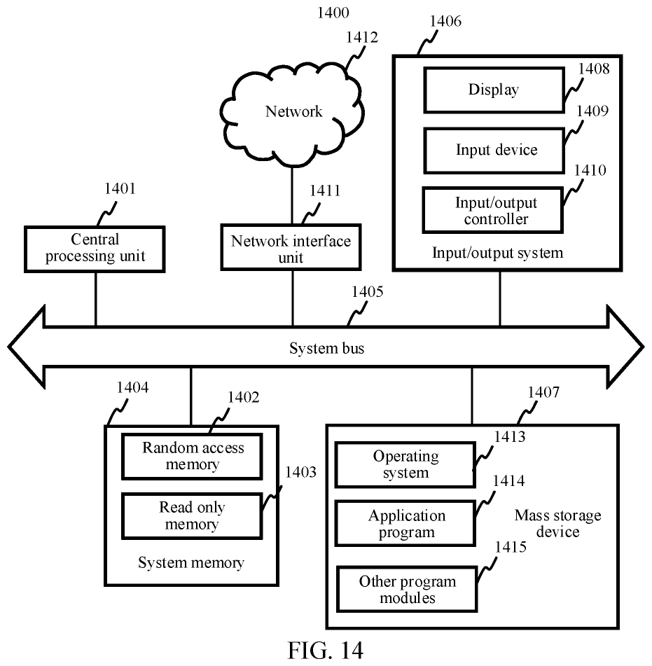

FIG. 14 is a schematic diagram of a structure of a computer device provided according to an example embodiment of this disclosure. The computer device may be a terminal or a server. In some examples, the computer device 1400 includes a central processing unit (CPU) 1401, a system memory 1404 including a random access memory 1402 and a read only memory 1403, and a system bus 1405 connecting the system memory 1404 and the central processing unit 1401. The computer device 1400 further includes a basic input/output (I/O) system 1406 assisting in information transmission between devices in a computer, and a mass storage device 1407 configured to store an operating system 1413, an application 1414, and other program modules 1415.

In some embodiments, the basic I/O system 1406 includes a display 1408 configured to display information and an input device 1409 such as a mouse or a keyboard that is configured for inputting information by a user. The display 1408 and the input device 1409 are both connected to the CPU 1401 through an input/output controller 1410 connected to the system bus 1405. The basic I/O system 1406 may further include the input and output controller 1410 to be configured to receive and process inputs from a plurality of other devices such as a keyboard, a mouse, and an electronic stylus. Similarly, the input/output controller 1410 further provides an output to a display screen, a printer, or another type of output device.

The mass storage device 1407 is connected to the CPU 1401 by using a mass storage controller (not shown) connected to the system bus 1405. The mass storage device 1407 and a computer-readable medium associated with the mass storage device provide non-volatile storage for the computer device 1400. That is, the mass storage device 1407 may include a computer-readable medium (not shown) such as a hard disk or a driver.

In general, the computer-readable medium may include a computer storage medium and a communication medium. The computer storage medium includes volatile and non-volatile media, and removable and non-removable media implemented by using any method or technology configured for storing information such as computer-readable instructions, data structures, program modules, or other data. The computer storage medium includes a random access memory (RAM), a read only memory (ROM), a flash memory or another solid-state memory, a compact disc read-only memory (CD-ROM), a digital versatile disc (DVD) or another optical memory, a magnetic cassette, a magnetic tape, a disk memory, or another magnetic storage device. It is noted that the computer storage medium is not limited to the foregoing several types. The system memory 1404 and the mass storage device 1407 may be collectively referred to as a memory.

The memory has one or more programs stored therein, the one or more programs being configured to be executed by one or more CPUs 1401, and the one or more programs including instructions for implementing the foregoing method. The CPU 1401 executes the one or more programs to implement the method provided in the foregoing method embodiments.

According to the embodiments of this disclosure, the computer device 1400 may further be connected, through a network such as the Internet, to a remote computer on the network and run. To be specific, the computer device 1400 may be connected to a network 1412 by using a network interface unit 1411 connected to the system bus 1405, or may be connected to another type of network or a remote computer system (not shown) by using a network interface unit 1411.

This disclosure further provides a computer-readable storage medium. The storage medium has at least one program stored therein, the at least one program being loaded and executed by a processor to implement the molecular generation method provided in any one of the foregoing embodiments.

The embodiments of this disclosure provide a computer program product or a computer program. The computer program product or computer program includes at least one program. The at least one program is stored in a computer-readable storage medium. A processor of a computer device reads the at least one program from the computer-readable storage medium, and the processor executes the at least one program to cause the computer device to perform the molecular generation method provided in the foregoing aspects.

It is noted that all or some of the operations of the methods in the embodiments may be implemented by a program instructing relevant hardware. The program may be stored in a computer-readable storage medium. The computer-readable storage medium may be the computer-readable storage medium included in the memory in the foregoing embodiment, or may be a computer-readable storage medium that exists independently and that is not installed into a terminal.

In some embodiments, the computer-readable storage medium may include: a ROM, a RAM, a solid state drive (SSD), an optical disc, or the like. The RAM may include a resistance random access memory (ReRAM) and a dynamic random access memory (DRAM). The sequential numbers of the foregoing embodiments of this disclosure are merely for description purpose but do not imply the preference of the embodiments.

It is noted that all or some of the operations of the foregoing embodiments may be implemented by hardware, or may be implemented by a program instructing relevant hardware. The program may be stored in a computer-readable storage medium. The storage medium mentioned above may be a ROM, a magnetic disk, an optical disc, or the like.

“A plurality of” mentioned herein means two or more. The term “and/or” describes an association relationship of associated objects, representing that three relationships may exist. For example, A and/or B may represent three situations: A exists alone; A and B exist simultaneously; and B exists alone. The character “/” usually indicates an “or” relationship between associated objects. Terms “first”, “second”, and the like mentioned herein are used to distinguish between similar objects, and are not intended to limit a specific order or sequence. In addition, the operation numbers described in this specification are examples to show an execution sequence of the operations. In some other embodiments, the operations may not be performed according to the number sequence. For example, two operations with different numbers may be performed simultaneously, or two operations with different numbers may be performed according to a sequence contrary to the sequence shown in the figure. This is not limited in the embodiments of this disclosure.

All the technical features of the above embodiments can be combined in different manners to form other embodiments. For the sake of brevity, all possible combinations of all the technical features in the foregoing embodiments are not described. However, these technical features shall all be considered to fall within the scope of this specification as long as there is no contradiction in their combinations.

One or more modules, submodules, and/or units of the apparatus can be implemented by processing circuitry, software, or a combination thereof, for example. The term module (and other similar terms such as unit, submodule, etc.) in this disclosure may refer to a software module, a hardware module, or a combination thereof. A software module (e.g., computer program) may be developed using a computer programming language and stored in memory or non-transitory computer-readable medium. The software module stored in the memory or medium is executable by a processor to thereby cause the processor to perform the operations of the module. A hardware module may be implemented using processing circuitry, including at least one processor and/or memory. Each hardware module can be implemented using one or more processors (or processors and memory). Likewise, a processor (or processors and memory) can be used to implement one or more hardware modules. Moreover, each module can be part of an overall module that includes the functionalities of the module. Modules can be combined, integrated, separated, and/or duplicated to support various applications. Also, a function being performed at a particular module can be performed at one or more other modules and/or by one or more other devices instead of or in addition to the function performed at the particular module. Further, modules can be implemented across multiple devices and/or other components local or remote to one another. Additionally, modules can be moved from one device and added to another device, and/or can be included in both devices.

The use of “at least one of” or “one of” in the disclosure is intended to include any one or a combination of the recited elements. For example, references to at least one of A, B, or C; at least one of A, B, and C; at least one of A, B, and/or C; and at least one of A to C are intended to include only A, only B, only C or any combination thereof. References to one of A or B and one of A and B are intended to include A or B or (A and B). The use of “one of” does not preclude any combination of the recited elements when applicable, such as when the elements are not mutually exclusive.

The foregoing descriptions are merely non-limiting examples of embodiments of this disclosure, and are not intended to limit this disclosure. Any modification, equivalent replacement, or improvement made within the spirit and principle of this disclosure shall fall within the scope of this disclosure.

What is claimed is:

1. A method of molecular generation, the method comprising:
generating initial hierarchical embeddings, the initial hierarchical embeddings comprising an initial molecular graph embedding of an initial molecular graph, an initial subgraph embedding of one or more initial subgraphs of the initial molecular graph, and an initial atomic embedding of initial atoms, the initial molecular graph including initial nodes and initial edges, the initial nodes representing the initial atoms, the initial edges representing chemical bonds connecting the initial atoms, and each of the one or more initial subgraphs representing a regional molecular structure of the initial molecular graph;
removing noises from the initial hierarchical embeddings to obtain generated hierarchical embeddings, the noises being predicted based on a machine learning model that includes learned relationships among molecular graphs, subgraphs, and atoms, the generated hierarchical embeddings including a generated molecular graph embedding, a generated subgraph embedding, and a generated atomic embedding of generated hierarchical embeddings; and
decoding the generated hierarchical embeddings into molecular structure information of a generated molecule, the molecular structure information comprising atom types of used atoms in the generated molecule and chemical bond types of inter-atomic chemical bonds connecting the used atoms in the generated molecule.

2. The method according to claim 1, wherein the removing the noises comprises:
predicting the noises based on at least two of the initial molecular graph embedding, the initial subgraph embedding, and the initial atomic embedding.

3. The method according to claim 1, wherein the generating the initial hierarchical embeddings comprises one of:
sampling a preset candidate molecular graph embedding set, a preset candidate subgraph embedding set, and a preset candidate atomic embedding set respectively according to a Gaussian distribution, to obtain the initial hierarchical embeddings; and
sampling respective preset parameter value sets of at least two parameters according to the Gaussian distribution to obtain sampled values of at least the two parameters, generating initial molecular structure information based on the sampled values of at least the two parameters, and transforming the initial molecular structure information into the initial hierarchical embeddings.

4. The method according to claim 1, wherein the removing the noises comprises:
obtaining first intermediate hierarchical embeddings by removing first noises from initial hierarchical embeddings;
obtaining (n+1)^{th} intermediate hierarchical embeddings by removing (n+1)^{th} noises from n^{th} intermediate hierarchical embeddings, the n^{th} intermediate hierarchical embeddings comprising an n^{th} intermediate molecular graph embedding, an n^{th} intermediate subgraph embedding, and an n^{th} intermediate atomic embedding; and
obtaining the generated hierarchical embeddings by removing n^{th} noises from (N−1)^{th} intermediate hierarchical embeddings;
N being a preset total number of instances of denoising, and n being a positive integer greater than 0 and less than N.

5. The method according to claim 4, wherein:
the machine learning model comprises a hierarchical denoising network;
the hierarchical denoising network comprises a first multilayer perceptron network, a second multilayer perceptron network, and at least a hierarchical block located between the first multilayer perceptron network and the second multilayer perceptron network; and
the removing the (n+1)^{th} noises from n^{th} intermediate hierarchical embeddings comprises:
transforming the n^{th} intermediate hierarchical embeddings into n^{th} intermediate hierarchical variables according to the first multilayer perceptron network, the n^{th} intermediate hierarchical variables comprising at least an n^{th} intermediate molecular graph variable, an n^{th} intermediate subgraph variable, and an n^{th} intermediate atomic variable;
updating the n^{th} intermediate hierarchical variables according to at least the hierarchical block based on a relationship among at least the n^{th} intermediate molecular graph variable, the n^{th} intermediate subgraph variable, and the n^{th} intermediate atomic variable, to obtain updated n^{th} intermediate hierarchical variables; and
predicting the (n+1)^{th} noises according to the second multilayer perceptron network based on the updated n^{th} intermediate hierarchical variables.

6. The method according to claim 5, wherein:
the hierarchical denoising network further comprises a sixth multilayer perceptron and a property attention network; and
the obtaining the (n+1)^{th} intermediate hierarchical embeddings comprises:
removing the (n+₁)^{th} noises from the n^{th} intermediate hierarchical embeddings to obtain denoised n^{th} intermediate hierarchical embeddings;
transforming a target molecular property embedding into at least a variable in a target property hidden space according to the sixth multilayer perceptron; and
updating the denoised n^{th} intermediate hierarchical embeddings according to the property attention network based on at least the variable in the target property hidden space to obtain the (n+1)^{th} intermediate hierarchical embeddings.

7. The method according to claim 5, wherein:
at least the hierarchical block comprises a plurality of hierarchical blocks connected in series that updates the n^{th} intermediate hierarchical variables in series, and
the updating the n^{th} intermediate hierarchical variables comprises:
using a first hierarchical block in the plurality of hierarchical blocks to: update the n^{th} intermediate atomic variable based on the n^{th} intermediate atomic variable and the n^{th} intermediate subgraph variable that are outputted by the first multilayer perceptron network, update the n^{th} intermediate subgraph variable based on the n^{th} intermediate subgraph variable and the n^{th} intermediate atomic variable, and update the n^{th} intermediate molecular graph variable based on the n^{th} intermediate subgraph variable, the n^{th} intermediate atomic variable, and the n^{th} intermediate molecular graph variable to generate a first updated n^{th} intermediate subgraph variable, a first updated n^{th} intermediate atomic variable, and a first update n^{th} intermediate molecular graph variable;
using an (m+1)^{th} hierarchical block in the plurality of hierarchical blocks to: receive an m^{th} updated n^{th} intermediate atomic variable, an m^{th} updated n^{th} intermediate subgraph variable and an m^{th} updated n^{th} intermediate molecular graph variable that are output by an m^{th} hierarchical block in the plurality of hierarchical blocks, update the m^{th} updated n^{th} intermediate atomic variable based on the m^{th} updated n^{th} intermediate atomic variable and the m^{th} updated n^{th} intermediate subgraph variable, update the m^{th} updated n^{th} intermediate subgraph variable based on the m^{th} updated n^{th} intermediate subgraph variable and the m^{th} updated n^{th} intermediate atomic variable, and update the n^{th} intermediate molecular graph variable based on the n^{th} intermediate subgraph variable, the n^{th} intermediate atomic variable, and the n^{th} intermediate molecular graph variable; and
outputting, to the second multilayer perceptron network after an updating by an M^{th} hierarchical block of the plurality of hierarchical blocks, the updated n^{th} intermediate hierarchical variables including an M^{th} updated n^{th} intermediate atomic variable, an M^{th} updated n^{th} intermediate subgraph variable, and an M^{th} updated n^{th} intermediate molecular graph variable;
M being a total number of the plurality of hierarchical blocks, and m being an integer greater than 0 and less than M.

8. The method according to claim 7, wherein:
the (m+1)^{th} hierarchical block comprises a third multilayer perceptron; and
the using the (m+1)^{th} hierarchical block comprises:
transforming, according to the third multilayer perceptron, the m^{th} updated n^{th} intermediate atomic variable to obtain a first transformed variable;
transforming, according to the third multilayer perceptron, the m^{th} updated n^{th} intermediate subgraph variable to obtain a second transformed variable;
fusing the first transformed variable with the second transformed variable to obtain a first fused variable; and
transforming the first fused variable according to the third multilayer perceptron to obtain an (m+1)^{th} updated n^{th} intermediate atomic variable.

9. The method according to claim 7, wherein:
the (m+1)^{th} hierarchical block comprises a fourth multilayer perceptron and an attention network; and
the using the (m+1)^{th} hierarchical block comprises:
determining a first attention score according to the attention network based on the m^{th} updated n^{th} intermediate atomic variable and the m^{th} updated n^{th} intermediate subgraph variable;
weighting, based on the first attention score, the m^{th} updated n^{th} intermediate atomic variable, to obtain a weighted m^{th} updated n^{th} intermediate atomic variable;
transforming, according to the fourth multilayer perceptron, the m^{th} updated n^{th} intermediate subgraph variable, to obtain a third transformed variable;
fusing the third transformed variable with the weighted m^{th} updated n^{th} intermediate atomic variable to obtain a second fused variable; and
transforming the second fused variable according to the fourth multilayer perceptron to obtain an (m+1)^{th} updated n^{th} intermediate subgraph variable.

10. The method according to claim 7, wherein:
the (m+1)^{th} hierarchical block comprises a fifth multilayer perceptron and a neighborhood aggregator; and
the using the (m+1)^{th} hierarchical block comprises:
transforming, according to the fifth multilayer perceptron, the m^{th} updated n^{th} intermediate molecular graph variable, to obtain a fourth transformed variable;
performing, according to the neighborhood aggregator, neighborhood aggregation on the m^{th} updated n^{th} intermediate atomic variable to obtain a first aggregated variable;
performing, according to the neighborhood aggregator, neighborhood aggregation on the m^{th} updated n^{th} intermediate subgraph variable to obtain a second aggregated variable;
fusing the fourth transformed variable, the first aggregated variable, and the second aggregated variable to obtain a third fused variable; and
transforming the third fused variable according to the fifth multilayer perceptron to obtain an (m+1)^{th} updated n^{th} intermediate molecular graph variable.

11. The method according to claim 6, wherein the updating the denoised n^{th} intermediate hierarchical embeddings comprises:
determining, according to the property attention network, second attention scores of the variable in the target property hidden space and elements in the denoised n^{th} intermediate hierarchical embeddings, the second attention scores representing associations between the elements and the variable in target property hidden space, each element comprising at least one of: an embedding of an atomic in the n^{th} intermediate atomic embedding, an embedding of a subgraph in the n^{th} intermediate subgraph embedding, an embedding of a node in the n^{th} intermediate molecular graph embedding and an embedding of an edge in the n^{th} intermediate molecular graph embedding; and
generating the (n+1)^{th} intermediate hierarchical embeddings by weighting at least one element in the denoised n^{th} intermediate hierarchical embeddings based on the second attention scores.

12. The method according to claim 4, wherein:
the method further comprises:
generating a noise; and
performing noise addition to the n^{th} intermediate hierarchical embeddings based on the generated noise to obtain noise-added n^{th} intermediate hierarchical embeddings; and
the obtaining the (n+1)^{th} intermediate hierarchical embeddings comprises:
denoising the noise-added n^{th} intermediate hierarchical embeddings.

13. The method according to claim 1, wherein:
the machine learning model comprises an encoding network, a hierarchical denoising network, and a decoding network; and
the method further comprises:
training the encoding network and the decoding network based on first sample molecular structure information of a first sample molecule; and
training the hierarchical denoising network and the encoding network based on second sample molecular structure information of a second sample molecule, the encoding network encoding the second sample molecular structure information to obtain second sample hierarchical embeddings, and the hierarchical denoising network being trained to denoise the second sample hierarchical embeddings.

14. The method according to claim 13, wherein the training the encoding network and the decoding network comprises:
inputting the first sample molecular structure information to the encoding network to obtain first sample hierarchical embeddings, the first sample hierarchical embeddings comprising a first sample graph embedding, a first sample subgraph embedding, and a first sample atomic embedding;
decoding the first sample hierarchical embeddings according to the decoding network to obtain estimated molecular structure information of an estimated sample molecule;
determining a first reconstruction loss based on a difference between the estimated molecular structure information and the first sample molecular structure information;
determining a Kullback-Leibler divergence loss based on a difference between the first sample hierarchical embeddings and a standard Gaussian distribution;
determining a sum of the first reconstruction loss and the Kullback-Leibler divergence loss as a total estimated loss; and
training the encoding network and the decoding network based on the total estimated loss.

15. The method according to claim 13, wherein the training the hierarchical denoising network and the encoding network comprises:
inputting the second sample molecular structure information to the encoding network to obtain the second sample hierarchical embeddings outputted by the encoding network, the second sample hierarchical embeddings comprising a second sample graph embedding, a second sample subgraph embedding, and a second sample atomic embedding;
generating sample noises corresponding to the second sample graph embedding, the second sample subgraph embedding, and the second sample atomic embedding;
adding the sample noises on the second sample hierarchical embeddings to obtain second sample noisy hierarchical embeddings, the second sample noisy hierarchical embeddings comprising a second sample noisy graph embedding, a second sample noisy subgraph embedding, and a second sample noisy atomic embedding;
performing noise prediction on the second sample noisy hierarchical embeddings according to the hierarchical denoising network to obtain predicted noises;
determining a noise prediction loss based on a difference between the predicted noises and the sample noises; and
training the hierarchical denoising network and the encoding network based on the noise prediction loss.

16. The method according to claim 13, wherein the training the hierarchical denoising network and the encoding network comprises:
training the hierarchical denoising network and the encoding network based on the second sample molecular structure information and a sample molecular property of the second sample molecule.

17. An apparatus of molecular generation, comprising processing circuitry configured to:
generate initial hierarchical embeddings, the initial hierarchical embeddings comprising an initial molecular graph embedding of an initial molecular graph, an initial subgraph embedding of one or more initial subgraphs of the initial molecular graph, and an initial atomic embedding of initial atoms, the initial molecular graph including initial nodes and initial edges, the initial nodes representing the initial atoms, the initial edges representing chemical bonds connecting the initial atoms, and each of the one or more initial subgraphs representing a regional molecular structure of the initial molecular graph;
remove noises from the initial hierarchical embeddings to obtain generated hierarchical embeddings, the noises being predicted based on a machine learning model that includes learned relationships among molecular graphs, subgraphs, and atoms, the generated hierarchical embeddings including a generated molecular graph embedding, a generated subgraph embedding, and a generated atomic embedding of generated hierarchical embeddings; and
decode the generated hierarchical embeddings into molecular structure information of a generated molecule, the molecular structure information comprising atom types of used atoms in the generated molecule and chemical bond types of inter-atomic chemical bonds connecting the used atoms in the generated molecule.

18. The apparatus according to claim 17, wherein the processing circuitry is configured to:
predict the noises based on at least two of the initial molecular graph embedding, the initial subgraph embedding, and the initial atomic embedding.

19. The apparatus according to claim 17, wherein the processing circuitry is configured to perform at least one of:
sampling a preset candidate molecular graph embedding set, a preset candidate subgraph embedding set, and a preset candidate atomic embedding set respectively according to a Gaussian distribution, to obtain the initial hierarchical embeddings; and
sampling respective preset parameter value sets of at least two parameters according to the Gaussian distribution to obtain sampled values of at least the two parameters, generating initial molecular structure information based on the sampled values of at least the two parameters, and transforming the initial molecular structure information into the initial hierarchical embeddings.

20. A non-transitory computer-readable storage medium storing instructions which when executed by at least one processor cause the at least one processor to perform:
generating initial hierarchical embeddings, the initial hierarchical embeddings comprising an initial molecular graph embedding of an initial molecular graph, an initial subgraph embedding of one or more initial subgraphs of the initial molecular graph, and an initial atomic embedding of initial atoms, the initial molecular graph including initial nodes and initial edges, the initial nodes representing the initial atoms, the initial edges representing chemical bonds connecting the initial atoms, and each of the one or more initial subgraphs representing a regional molecular structure of the initial molecular graph;
removing noises from the initial hierarchical embeddings to obtain generated hierarchical embeddings, the noises being predicted based on a machine learning model that includes learned relationships among molecular graphs, subgraphs, and atoms, the generated hierarchical embeddings including a generated molecular graph embedding, a generated subgraph embedding, and a generated atomic embedding of generated hierarchical embeddings; and
decoding the generated hierarchical embeddings into molecular structure information of a generated molecule, the molecular structure information comprising atom types of used atoms in the generated molecule and chemical bond types of inter-atomic chemical bonds connecting the used atoms in the generated molecule.
